(19) United States
(12) Patent Application Publication
(10) Pub. No.: US 2026/0256762 A1
(43) Pub. Date: Sep. 3, 2026

(54) Aromatic Amide Derivative, And Preparation Method Therefor And Medical Use Thereof

(71) Applicants: ZHEJIANG HISUN PHARMACEUTICAL CO., LTD., Taizhou, Zhejiang (CN); SHANGHAI ARYL PHARMTECH CO., LTD., Shanghai (CN)

(72) Inventors: Zongxing Qiu, Shanghai (CN); Panpan ZHANG, Taizhou, Zhejiang (CN); Hai CAO, Shanghai (CN); Jilei PENG, Shanghai (CN); Zefeng WANG, Shanghai (CN); Cheng YE, Shanghai (CN); Daiwang XU, Shanghai (CN); Xiaojie XU, Taizhou, Zhejiang (CN)

(73) Assignees: ZHEJIANG HISUN PHARMACEUTICAL CO., LTD., Taizhou, Zhejiang (CN); SHANGHAI ARYL PHARMTECH CO., LTD., Shanghai (CN)

(21) Appl. No.: 19/143,686

(22) PCT Filed: Jan. 4, 2024
(86) PCT No.: PCT/CN2024/070546

§ 371 Date: Jun. 26, 2025

(30) Foreign Application Priority Data
Jan. 5, 2023 (CN) 202310013810.X

(51) Int. Cl. A61K 31/4545; A61K 31/438; A61K 31/501; A61K 31/5377; A61P 35/00; C07D 221/20; C07D 401/12; C07D 401/14

(52) U.S. Cl. CPC A61K 31/4545; A61K 31/438; A61K 31/501; A61K 31/5377; A61P 35/00; C07D 221/20; C07D 401/12; C07D 401/14

(57) ABSTRACT

An aromatic amide derivative, a preparation method therefor, and use of a pharmaceutical composition containing the derivative in medicine. Specifically, the present invention relates to an aromatic amide derivative as shown by general formula (I), a preparation method therefor, a pharmaceutically acceptable salt thereof, and use thereof as a therapeutic agent, particularly as a KIF18A inhibitor, wherein the definition of each substituent in general formula (I) is the same as the definition in the description.

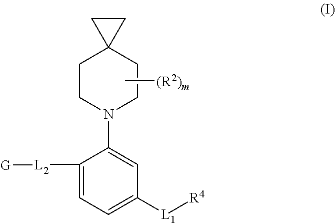

### TECHNICAL FIELD

The present invention relates to an aromatic amide derivative, a preparation method therefor, a pharmaceutical composition comprising the derivative, and use thereof as a therapeutic agent, particularly as a KIF18A inhibitor.

### BACKGROUND

Kinesin molecules are motor proteins with microtubules as orbits thereof, which play an important role in the process of organelle migration, tissue and organ development, signal transduction, mitosis and meiosis, etc. Many microtubule-associated proteins (MAP) of a kinesin-8 family play a role in regulating the dynamic instability of the microtubules by influencing the polymerization and depolymerization of the microtubules. KIF18A, a member of kinesin-8 family, may move towards a positive pole with the microtubules as an orbit, and tends to combine with the longer microtubules. The activity of KIF18A is length-dependent and affects a length of a spindle, which may ensure the timely and successfully completion of sister chromosome alignment, and the functions in different species are very similar and conservative.

KIF18A is a molecular motor protein that moves towards a positive pole end of the microtubule, regulates a middle plate assembly of chromosomes by affecting the dynamic instability of the end of the microtubule, and plays a role in mitosis. At the late stage of mitosis, the protein is ubiquitinated and degraded, which ensures the accurate separation of chromosomes during mitosis and promotes the successfully completion of mitosis and cytokinesis. At the early stage of mitosis, it is necessary for KIF18A to locate at the positive pole end of microtubule near kinetochore to perform a function. Localization depends not only on the motor activity of an N-end thereof, but also a tail domain with microtubule binding ability plays a key role. KIF18A is also modified by reversible protein phosphorylation/dephosphorylation, but there is still a lack of systematic research on how the post-translation modification of the protein regulates the function of KIF18A. ERα, an estrogen receptor, can bind to KIF18A and promote transcription thereof. However, it is still unclear whether KIF18A is regulated by other transcription factors, so the research on a gene transcription regulation mechanism of KIF18A needs to be further studied. In the process of meiosis, cells without KIF18A will not be able to complete meiosis, which will lead to sperm formation disorder and testicular dysplasia in male animals.

A research has found that a KIF18A protein is highly expressed in many cancers, including but not limited to hepatocellular carcinoma, glioblastoma, colon cancer, breast cancer, lung cancer, cholangiocarcinoma, pancreatic cancer, prostate cancer, bladder cancer, head cancer, neck cancer, cervical cancer, ovarian cancer, synovial sarcoma, rhabdomyosarcoma, etc., which indicates that KIF18A is closely related to the occurrence and development of tumors and may be a target of molecular diagnosis and treatment of various tumors. The expression of KIF18A is related to the development of a clinical colorectal cancer. The research has found that KIF18A can induce Akt phosphorylation, and knocking out KIF18A in mice may obviously promote cell apoptosis. Therefore, it is speculated that KIF18A may promote the occurrence and development of colorectal cancer by activating a PI3K-Akt signaling channel. KIF18A is also highly expressed in human breast cancer cells, and the overexpression thereof is related to the classification, migration and prognosis of breast tumors. The study of breast cancer cells showed that the overexpression of KIF18A would lead to the generation of multinucleated cells, but the low expression could greatly weaken the cell proliferation ability in vivo and in vitro, which was induced by the stabilization of microtubules at the ends of microtubules by KIF18A and the inactivation of a PI3K-Akt signal transduction pathway. In addition, KIF18A in the lung adenocarcinoma is up-regulated at the level of transcription and translation, and the abnormal expression of KIF18A is related to the clinical and pathological malignancy. KIF18 gene mutation can be observed in the lung adenocarcinoma, and the expression thereof is also regulated by a DNA copy number. KIF18A gene knockout can inhibit the proliferation of lung adenocarcinoma cells in vivo and in vitro, and induce apoptosis and G2/M phase arrest. Genes that are highly expressed at the same time as KIF18A are concentrated in a cell cycle signal channel, so it is of great clinical significance to study the mechanism of KIF18A in tumor.

There is no new drug on the market for inhibitors targeting KIF18A. At present, only AMG-650 from Amgen Company of the United States has entered clinical phase I. As a relatively advanced research direction, there is still a huge exploration space for the related research of a KIF18A target, so it is necessary to continue to study an action mechanism thereof and develop new inhibitors.

### SUMMARY

The present invention provides a compound as shown by general formula (I), or a stereoisomer or a tautomer thereof or a pharmaceutically acceptable salt thereof:

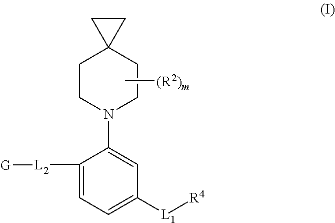

wherein,
G is selected from

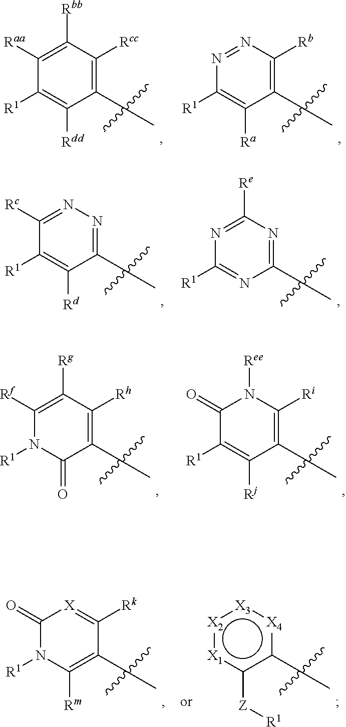

R^{aa}, R^{bb}, R^{cc} and R^{dd} are the same or different and are each independently selected from hydrogen atom, hydroxyl, cyano, amino, alkoxy or haloalkoxy, and R^{aa}, R^{bb}, R^{cc} and R^{dd} are not hydrogen atoms simultaneously;
R^{a}, R^{b}, R^{c}, R^{d}, R^{e}, R^{f}, R^{g}, R^{h}, Rⁱ, R^{j}, R^{k} and R^{m} are the same or different and are each independently selected from hydrogen atom, halogen, hydroxyl, cyano, alkyl or alkoxy; wherein the alkyl or alkoxy group is optionally further substituted by one or more substituents selected from halogen, hydroxyl, cyano, alkyl or alkoxy;
R^{ee} is selected from hydrogen atom or alkyl;
X is selected from N or CR^{ff}, and R^{ff} is selected from hydrogen atom, halogen, hydroxyl, cyano, alkyl, alkoxy, haloalkyl or haloalkoxy;
X₁, X₂, X₃ and X₄ are independently selected from CR^{gg} or N atom, and at most two atoms in X₁, X₂, X₃ and X₄ are N atoms at the same time, and R^{gg} is selected from hydrogen atom, halogen, hydroxyl, cyano, alkyl, alkoxy, haloalkyl or haloalkoxy;
Z is selected from a bond, —C(O)— or S(O)ᵣ;
L₁ is selected from a bond or C₁-C₆ alkylene, wherein the alkylene is optionally further substituted by one or more substituents selected from halogen, hydroxyl, cyano or alkoxy, and wherein one or more methylenes of the alkylenes are optionally substituted by one or more O, S(O)ᵣ, C(O) or NR^{A};
R^{A} is selected from hydrogen atom or alkyl;
L₂ is selected from

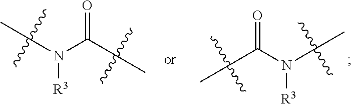

R³ is each independently selected from hydrogen atom or alkyl, wherein the alkyl is optionally further substituted by one or more substituents selected from halogen, hydroxyl, cyano or alkoxy; R³ is preferably a hydrogen atom;
R¹ is each independently selected from hydrogen atom, cyano, halogen, alkyl, cycloalkyl, heterocyclyl, aryl, heteroaryl, —OR⁵, —C(O)R⁵, —C(O)OR⁵, —NHC(O)R⁵, —NHC(O)OR⁵, —NR⁶R⁷, —C(O)NR⁶R⁷, —CH₂NHC(O)OR⁵, —CH₂NR⁶R⁷ or —S(O)ᵣR⁵, wherein the alkyl, cycloalkyl, heterocyclyl, aryl or heteroaryl is optionally further substituted by one or more substituents selected from hydroxyl, halogen, nitro, cyano, alkyl, alkoxy, haloalkyl, haloalkoxy, cycloalkyl, heterocyclyl, aryl, heteroaryl, ═O, —C(O)R⁸, —C(O)OR⁸, —OC(O)R⁸, —NR⁹R¹⁰, —C(O)NR⁹R¹⁰, —SO₂NR⁹R¹⁰ or —NR⁹C(O)R¹⁰;
R² is the same or different and is each independently selected from halogen, hydroxyl, cyano, alkyl or alkoxy; wherein alkyl or alkoxy is optionally further substituted by one or more substituents selected from halogen, hydroxyl, cyano, alkyl or alkoxy;
R⁴ is selected from hydrogen atom, cyano, halogen, alkyl, hydroxyl, cycloalkyl, heterocyclyl, aryl or heteroaryl; wherein alkyl, cycloalkyl, heterocyclyl, aryl or heteroaryl is optionally further substituted by one or more substituents selected from hydroxyl, halogen, nitro, cyano, alkyl, alkoxy, haloalkyl, haloalkoxy, cycloalkyl, halocycloalkyl, heterocyclyl, haloheterocyclyl, aryl, heteroaryl, haloheteroaryl, ═O, —C(O)R⁸, —C(O)OR⁸, —OC(O)R⁸, —NR⁹R¹⁰, —C(O)NR⁹R⁰, —SO₂NR⁹R¹⁰ or —NR⁹C(O)R¹⁰;
R⁵ is each independently selected from hydrogen atom, alkyl, cycloalkyl, heterocyclyl, aryl or heteroaryl; wherein alkyl, cycloalkyl, heterocyclyl, aryl or heteroaryl is optionally further substituted by one or more substituents selected from hydroxyl, halogen, nitro, cyano, alkyl, alkoxy, haloalkyl, haloalkoxy, cycloalkyl, heterocyclyl, aryl, heteroaryl, ═O, —C(O)R⁸, —C(O)OR⁸, —OC(O)R⁸, —NR⁹R¹⁰, —C(O)NR⁹R¹⁰, —SO₂NR⁹R¹⁰ or —NR⁹C(O)R¹⁰;
R⁶ and R⁷ are each independently selected from hydrogen atom, hydroxyl, halogen, alkyl, alkoxy, cycloalkyl, heterocyclyl, aryl or heteroaryl, wherein alkyl, alkoxy, cycloalkyl, heterocyclyl, aryl or heteroaryl is optionally further substituted by one or more substituents selected from hydroxyl, halogen, nitro, cyano, alkyl, alkoxy, cycloalkyl, heterocyclyl, aryl, heteroaryl, ═O, —C(O)R⁸, —C(O)OR⁸, —OC(O)R⁸, —NR⁹R¹⁰, —C(O)NR⁹R¹⁰, —SO₂NR⁹R¹⁰ or —NR⁹C(O)R¹⁰;
or, R⁶ and R⁷ and the atoms attached thereto form a 4-8-membered heterocyclyl group, wherein the 4-8-membered heterocyclyl group comprises one or more N, O or S(O)ᵣ, and the 4-8-membered heterocyclyl group is optionally further substituted by one or more substituents selected from hydroxyl, halogen, nitro, cyano, alkyl, alkoxy, cycloalkyl, heterocyclyl, aryl, heteroaryl, ═O, —C(O)R⁸, —C(O)OR⁸, —OC(O)R⁸, —NR⁹R¹⁰, —C(O)NR⁹R¹⁰, —SO₂NR⁹R¹⁰ or —NR⁹C(O)R¹⁰;
R⁸, R⁹ and R¹⁰ are each independently selected from hydrogen atom, alkyl, amino, cycloalkyl, heterocyclyl, aryl or heteroaryl, wherein alkyl, cycloalkyl, heterocyclyl, aryl or heteroaryl is optionally further substituted by one or more substituents selected from hydroxyl, halogen, nitro, amino, cyano, alkyl, alkoxy, cycloalkyl, heterocyclyl, aryl, heteroaryl, carboxyl or carboxylate.
m is 0, 1 or 2; m is preferably 0; and
r is independently 0, 1 or 2.

In a preferred embodiment of the present invention, the compound as shown by general formula (I), or the stereoisomer or the tautomer thereof or the pharmaceutically acceptable salt thereof is a compound as shown by general formula (II) or general formula (III), or a stereoisomer, a tautomer thereof or a pharmaceutically acceptable salt thereof:

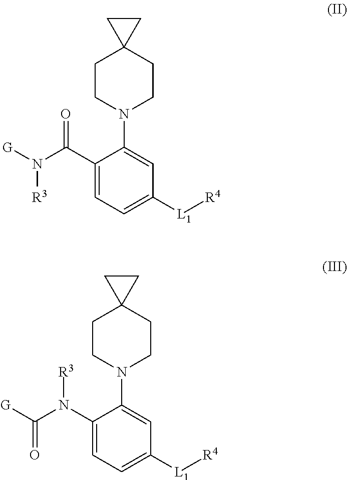

wherein, the definitions of G, R³, R⁴ and L₁ are as described in general formula (I).

In the preferred embodiment of the present invention, the compound as shown by general formula (I), general formula (II) or general formula (III), or the stereoisomer or the tautomer thereof or the pharmaceutically acceptable salt thereof, wherein G is selected from

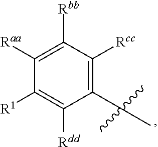

wherein,
R^{aa}, R^{bb}, R^{cc} and R^{dd} are selected from hydrogen atom, hydroxyl, cyano, amino, methoxy or trifluoromethoxy; and
R¹ is selected from phenyl, 5-6-membered heteroaryl, 3-10-membered heterocyclyl or C₃-C₁₀ cycloalkyl, wherein the phenyl, 5-6-membered heteroaryl, 3-10-membered heterocyclyl or C₃-C₁₀ cycloalkyl is optionally further substituted by one or more substituents selected from halogen, alkyl, haloalkyl, alkoxy or haloalkoxy.

In a preferred embodiment of the present invention, the compound as shown by general formula (I), general formula (II) or general formula (III), or the stereoisomer or the tautomer thereof or the pharmaceutically acceptable salt thereof, wherein G is selected from

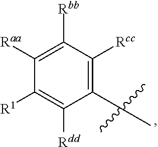

wherein,
R^{aa}, R^{bb}, R^{cc} and R^{dd} are selected from hydrogen atom, hydroxyl, cyano, amino group, methoxy or trifluoromethoxy; and
R¹ is selected from C₁₋₃ alkyl, wherein the C₁₋₃ alkyl is optionally further substituted by one or more substituents selected from cycloalkyl, halocycloalkyl, heterocyclyl, haloheterocyclyl, aryl, heteroaryl or haloheteroaryl.

In a preferred embodiment of the present invention, the compound as shown by general formula (I), general formula (II) or general formula (III), or the stereoisomer or the tautomer thereof or the pharmaceutically acceptable salt thereof, wherein R¹ is selected from

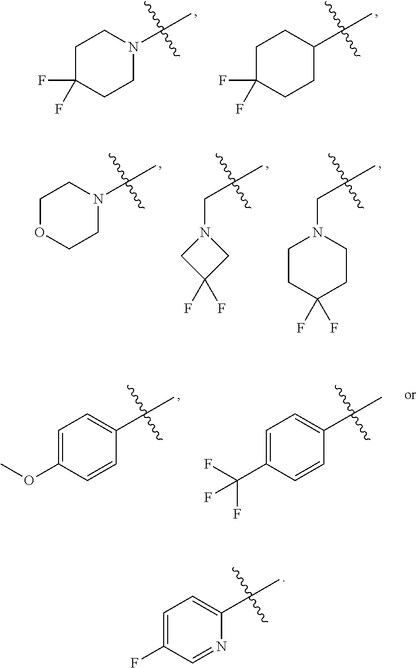

In a preferred embodiment of the present invention, the compound as shown by general formula (I), general formula (II) or general formula (III), or the stereoisomer or the tautomer thereof or the pharmaceutically acceptable salt thereof, wherein G is selected from

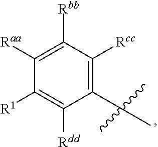

wherein,
R^{aa}, R^{bb}, R^{cc} and R^{dd} are selected from hydrogen atom, hydroxyl, cyano, amino or methoxy; and
R¹ is selected from 3-10-membered heterocyclyl or C₃-C₁₀ cycloalkyl, wherein the heterocyclyl or cycloalkyl is optionally further substituted by one or more halogens; and
R¹ is preferably selected from

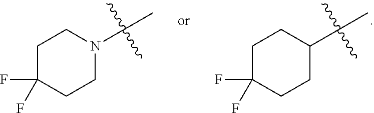 [SMILES: *N1CCC(F)(F)CC1 ; *C1CCC(F)(F)CC1]

In a preferred embodiment of the present invention, the compound as shown by general formula (I), general formula (II) or general formula (III), or the stereoisomer or the tautomer thereof or the pharmaceutically acceptable salt thereof, wherein G is selected from

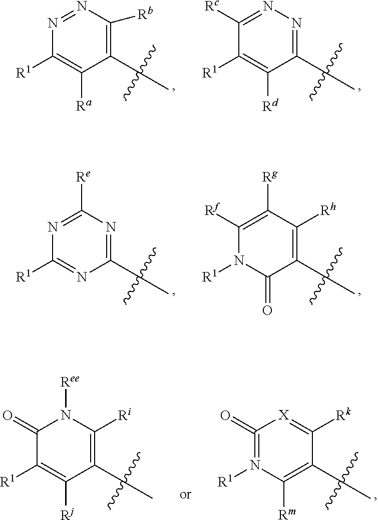

wherein,
R^{a}, R^{b}, R^{c}, R^{d}, R^{e}, R^{f}, R^{g}, R^{h}, Rⁱ, R^{j}, R^{k} and R^{m} are hydrogen atoms;
X is selected from N or CR^{ff}, R^{ee} and R^{ff} are independently selected from hydrogen atom or methyl;
each R¹ is independently selected from 3-10-membered heterocyclyl or C₃-C₁₀ cycloalkyl, wherein the heterocyclyl or cycloalkyl is optionally further substituted by one or more halogens; and
R¹ is preferably selected from

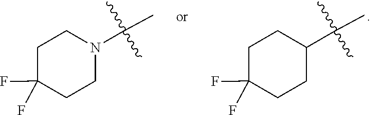 [SMILES: *N1CCC(F)(F)CC1 ; *C1CCC(F)(F)CC1]

In a preferred embodiment of the present invention, the compound as shown by general formula (I), general formula (II) or general formula (III), or the stereoisomer or the tautomer thereof or the pharmaceutically acceptable salt thereof, wherein G is selected from

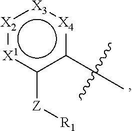

wherein,
Z is —C(O)—;
X₁, X₂, X₃ and X₄ are each independently selected from CR^{gg} or N atoms, and at most two atoms in X₁, X₂, X₃ and X₄ are N atoms at the same time, and R^{gg} is a hydrogen atom;
R¹ is selected from 3-10-membered heterocyclyl or C₃-C₁₀ cycloalkyl, wherein the heterocyclyl or cycloalkyl is optionally further substituted by one or more halogens; and
R¹ is preferably selected from

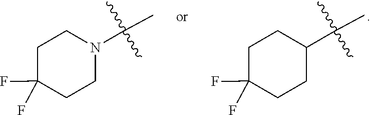 [SMILES: *N1CCC(F)(F)CC1 ; *C1CCC(F)(F)CC1]

In a preferred embodiment of the present invention, the compound as shown by general formula (I), general formula (II) or general formula (III), or the stereoisomer or the tautomer thereof or the pharmaceutically acceptable salt thereof, wherein
L₁ is selected from a bond or C₁-C₆ alkylene, wherein the alkylene is optionally further substituted by one or more hydroxyl, and wherein one or more methylene of the alkylene is optionally substituted by one or more O, S(O)ᵣ, C(O) or NR^{b};
r is 2; and
R^{b} is selected from the hydrogen atom or methyl.

In a preferred embodiment of the present invention, the compound as shown by general formula (I), general formula (II) or general formula (III), or the stereoisomer or the tautomer thereof or the pharmaceutically acceptable salt thereof, wherein
L₁ is selected from a bond, —NHSO₂CH₂CH₂—, —SO₂NHCH₂CH₂—, —SO₂—, —CH₂SO₂—, —NHSO₂—, —SO₂NH—, —NHC(CH₃)₂CH₂—, —C(O)NHCH₂CH₂—, —C(O)NHC(CH₃)₂CH₂—, —C(O)N(CH₃)CH₂CH₂—, —CH(CH₃)(OH)CH₂—, —NHSO₂CH(CH₃)CH₂—, —SO₂NHC(CH₃)₂CH₂—, —C(O)NH—, —NHCH₂CH₂ or —CH₂SO₂CH₂CH₂—.

In a preferred embodiment of the present invention, the compound as shown by general formula (I), general formula (II) or general formula (III), or the stereoisomer or the tautomer thereof or the pharmaceutically acceptable salt thereof, wherein
R⁴ is selected from hydrogen atom, hydroxyl, alkyl, heterocyclyl, cycloalkyl or heteroaryl, wherein the alkyl, heterocyclyl, cycloalkyl or heteroaryl is optionally further substituted by one or more substituents selected from hydroxyl or alkyl.

In a preferred embodiment of the present invention, the compound as shown by general formula (I), general formula (II) or general formula (III), or the stereoisomer or the tautomer thereof or the pharmaceutically acceptable salt thereof, wherein

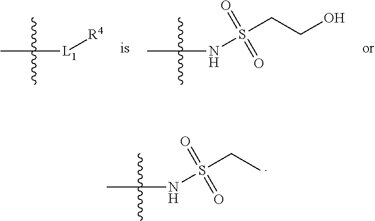

In a preferred embodiment of the present invention, the compound as shown by general formula (I), general formula (II) or general formula (III), or the stereoisomer or the tautomer thereof or the pharmaceutically acceptable salt thereof, wherein R³ is a hydrogen atom.

In a preferred embodiment of the present invention, the compound as shown by general formula is selected from:

| Compound | | |
|---|---|---|
| No. | Structure | Name |
| Example 1 | 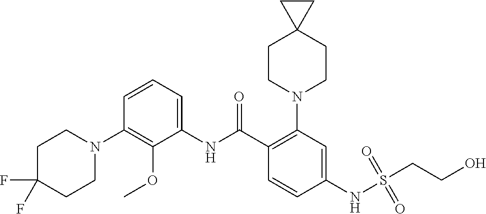 [SMILES: COc1c(NC(=O)c2ccc(NS(=O)(=O)CCO)cc2N2CCC3(CC2)CC3)cccc1N1CCC(F)(F)CC1] | N-(3-(4,4-difluoropiperidin- 1-yl)-2-methoxyphenyl)- 4-((2-hydroxyethyl) sulfonamido)-2- (6-azaspiro[2.5] octan-6-yl)benzamide |
| Example 2 | 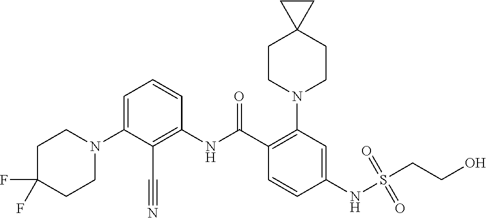 [SMILES: N#Cc1c(NC(=O)c2ccc(NS(=O)(=O)CCO)cc2N2CCC3(CC2)CC3)cccc1N1CCC(F)(F)CC1] | N-(2-cyano-3-(4,4- difluoropiperidin-1- yl)phenyl)-4- ((2-hydroxyethyl) sulfonamido)- 2-(6-azaspiro[2.5] octan-6-yl)benzamide |
| Example 3 | 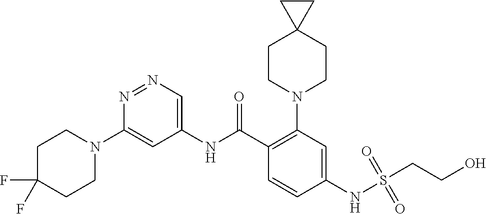 [SMILES: O=C(Nc1cnnc(N2CCC(F)(F)CC2)c1)c1ccc(NS(=O)(=O)CCO)cc1N1CCC2(CC1)CC2] | N-(6-(4,4-difluoropiperidin- 1-yl)pyridazin-4-yl)-4-((2- hydroxyethyl)sulfonamido)- 2-(6-azaspiro[2.5]octan- 6-yl)benzamide |
| Example 4 | 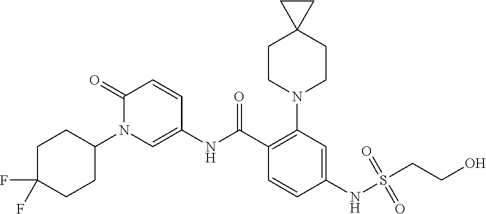 [SMILES: O=C(Nc1ccc(=O)n(C2CCC(F)(F)CC2)c1)c1ccc(NS(=O)(=O)CCO)cc1N1CCC2(CC1)CC2] | N-(1-(4,4-difluorocyclohexyl)- 6-oxo-1,6-dihydropyridin- 3-yl)-4-((2-hydroxyethyl) sulfonamido)-2-(6-azaspiro [2.5]octan-6-yl)benzamide |
| Example 5 | 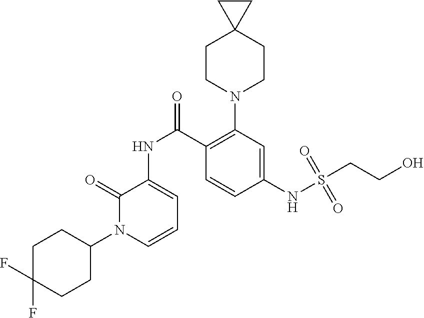 [SMILES: O=C(Nc1cccn(C2CCC(F)(F)CC2)c1=O)c1ccc(NS(=O)(=O)CCO)cc1N1CCC2(CC1)CC2] | N-(1-(4,4-difluorocyclohexyl)- 2-oxo-1,2-dihydropyridin- 3-yl)-4-((2-hydroxyethyl) sulfonamido)-2-(6- azaspiro[2.5]octan-6-yl) benzamide |
| Example 6 | 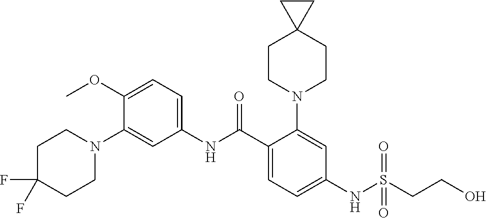 [SMILES: COc1ccc(NC(=O)c2ccc(NS(=O)(=O)CCO)cc2N2CCC3(CC2)CC3)cc1N1CCC(F)(F)CC1] | N-(3-(4,4-difluoropiperidin- 1-yl)-4-methoxyphenyl)- 4-((2-hydroxyethyl) sulfonamido)-2- (6-azaspiro[2.5] octan-6-yl)benzamide |
| Example 7 | 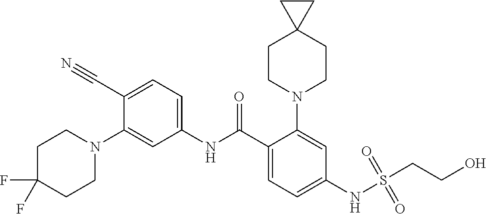 [SMILES: N#Cc1ccc(NC(=O)c2ccc(NS(=O)(=O)CCO)cc2N2CCC3(CC2)CC3)cc1N1CCC(F)(F)CC1] | N-(4-cyano-3- (4,4-difluoropiperidin- 1-yl)phenyl)-4- ((2-hydroxyethyl) sulfonamido)- 2-(6-azaspiro[2.5] octan-6-yl)benzamide |
| Example 8 | 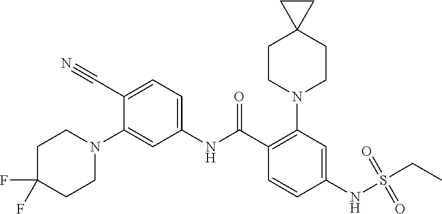 [SMILES: CCS(=O)(=O)Nc1ccc(C(=O)Nc2ccc(C#N)c(N3CCC(F)(F)CC3)c2)c(N2CCC3(CC2)CC3)c1] | N-(4-cyano-3- (4,4-difluoropiperidin- 1-yl)phenyl)-4- (ethylsulfonamido)-2-(6- azaspiro[2.5]octan-6-yl) benzamide |
| Example 9 | 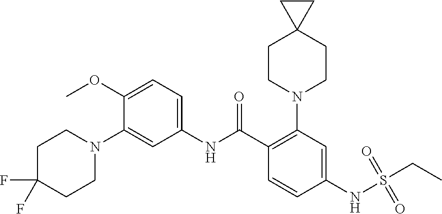 [SMILES: CCS(=O)(=O)Nc1ccc(C(=O)Nc2ccc(OC)c(N3CCC(F)(F)CC3)c2)c(N2CCC3(CC2)CC3)c1] | N-(3-(4,4-difluoropiperidin- 1-yl)-4-methoxyphenyl)- 4-(ethylsulfonamido)-2- (6-azaspiro[2.5]octan-6-yl) benzamide |
| Example 10 | 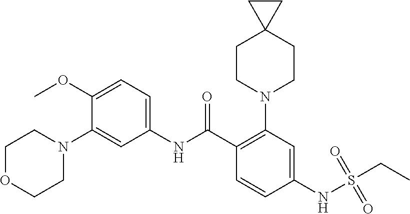 [SMILES: CCS(=O)(=O)Nc1ccc(C(=O)Nc2ccc(OC)c(N3CCOCC3)c2)c(N2CCC3(CC2)CC3)c1] | 4-(ethylsulfonamido)-N-(4- methoxy-3-morpholino phenyl)-2-(6-azaspiro[2.5] octan-6-yl)benzamide |
| Example 11 | 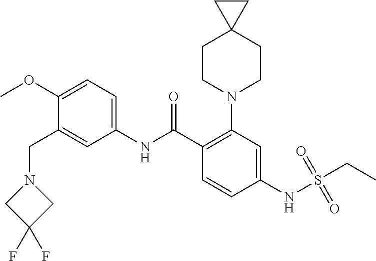 [SMILES: CCS(=O)(=O)Nc1ccc(C(=O)Nc2ccc(OC)c(CN3CC(F)(F)C3)c2)c(N2CCC3(CC2)CC3)c1] | N-(3-((3,3-difluoroazetidin- 1-yl)methyl)- 4-methoxyphenyl)- 4-(ethylsulfonamido)- 2-(6-azaspiro[2.5] octan-6-yl)benzamide |
| Example 12 | 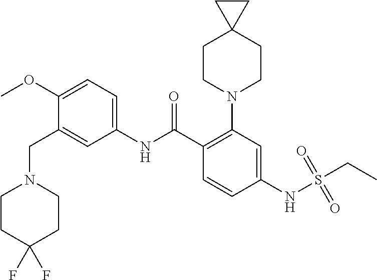 [SMILES: CCS(=O)(=O)Nc1ccc(C(=O)Nc2ccc(OC)c(CN3CCC(F)(F)CC3)c2)c(N2CCC3(CC2)CC3)c1] | N-(3-((4,4-difluoropiperidin- 1-yl)methyl)- 4-methoxyphenyl)- 4-(ethylsulfonamido)- 2-(6-azaspiro[2.5] octan-6-yl)benzamide |
| Example 13 | 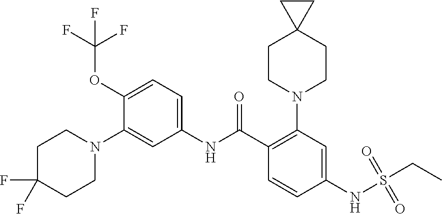 [SMILES: CCS(=O)(=O)Nc1ccc(C(=O)Nc2ccc(OC(F)(F)F)c(N3CCC(F)(F)CC3)c2)c(N2CCC3(CC2)CC3)c1] | N-(3-(4,4-difluoropiperidin- 1-yl)-4-(trifluoromethox y)phenyl)-4-(ethylsulfonamido)- 2-(6-azaspiro[2.5] octan-6-yl)benzamide |
| Example 14 | 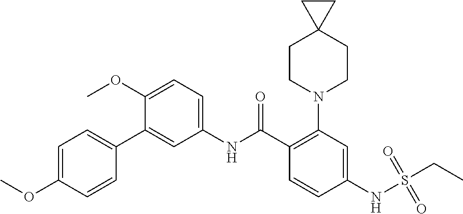 [SMILES: CCS(=O)(=O)Nc1ccc(C(=O)Nc2ccc(OC)c(-c3ccc(OC)cc3)c2)c(N2CCC3(CC2)CC3)c1] | N-(4′,6-dimethoxy-[1,1′- biphenyl]-3-yl)-4- (ethylsulfonamido)- 2-(6-azaspiro[2. 5]octan-6-yl)benzamide |
| Example 15 | 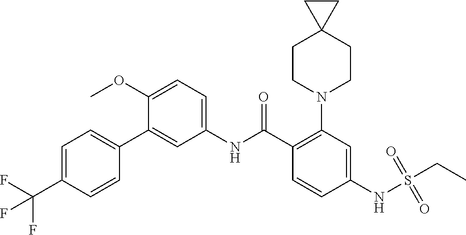 [SMILES: CCS(=O)(=O)Nc1ccc(C(=O)Nc2ccc(OC)c(-c3ccc(C(F)(F)F)cc3)c2)c(N2CCC3(CC2)CC3)c1] | 4-(ethylsulfonamido)-N-(6- methoxy-4′-(trifluoromethyl)- [1,1′-biphenyl]-3-yl)- 2-(6-azaspiro[2.5]octan-6- yl)benzamide |
| Example 16 | 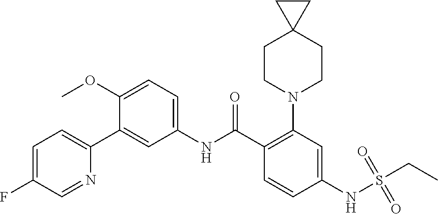 [SMILES: CCS(=O)(=O)Nc1ccc(C(=O)Nc2ccc(OC)c(-c3ccc(F)cn3)c2)c(N2CCC3(CC2)CC3)c1] | 4-(ethylsulfonamido)-N-(3- (5-fluoropyridine-2-yl)-4- methoxyphenyl)-2-(6-azaspiro [2.5]octan-6-yl) benzamide |

or the stereoisomer or the tautomer thereof or the pharmaceutically acceptable salt thereof.

Note: if there is a difference between the drawn structure and the given name of the structure, the drawn structure will be given greater weight.

Furthermore, the present invention provides a pharmaceutical composition, wherein the pharmaceutical composition comprises an effective dose of the compound as shown by general formula (I), general formula (II) or general formula (III), or the stereoisomer or the tautomer thereof or the pharmaceutically acceptable salt thereof, and a pharmaceutically acceptable carrier, an excipient or a composition thereof.

The present invention provides use of the compound as shown by general formula (I), general formula (II) or general formula (III), the stereoisomer or the tautomer thereof or the pharmaceutically acceptable salt thereof, or the pharmaceutical composition in the preparation of a KIF18A inhibitor.

The present invention further provides use of the compound as shown by general formula (I), general formula (II) or general formula (III), the stereoisomer or the tautomer thereof or the pharmaceutically acceptable salt thereof, or the pharmaceutical composition in the preparation of a medicament for treating a disease mediated by KIF18A, wherein the disease mediated by KIF18A is preferably a cancer; wherein the disease mediated by KIF18A is selected from hepatocellular carcinoma, glioblastoma, colon cancer, breast cancer, lung cancer, cholangiocarcinoma, pancreatic cancer, prostate cancer, bladder cancer, head cancer, neck cancer, cervical cancer, ovarian cancer, synovial sarcoma, rhabdomyosarcoma, colorectal cancer and lung adenocarcinoma.

The present invention further provides use of the compound as shown by general formula (I), general formula (II) or general formula (III), the stereoisomer or tautomer thereof or the pharmaceutically acceptable salt thereof, or the pharmaceutical composition in the preparation of a medicament for treating a cancer.

The present invention provides use of the compound as shown by general formula (I), general formula (II) or general formula (III), the stereoisomer or tautomer thereof or the pharmaceutically acceptable salt thereof, or the pharmaceutical composition in the preparation of a medicament for treating hepatocellular carcinoma, glioblastoma, colon cancer, breast cancer, lung cancer, cholangiocarcinoma, pancreatic cancer, prostate cancer, bladder cancer, head cancer, neck cancer, cervical cancer, ovarian cancer, synovial sarcoma, rhabdomyosarcoma, colorectal cancer and lung adenocarcinoma.

### DETAILED DESCRIPTION OF THE PRESENT INVENTION

Unless stated to the contrary, some terms used in the description and claims of the present invention are defined as follows:

“Alkyl”, when taken as a group or part of a group, refers to an aliphatic hydrocarbon group including a C₁-C₂₀ straight chain or a branched chain, preferably C₁-C₁₀ alkyl, more preferably C₁-C₆ alkyl. Examples of the alkyl group include, but are not limited to, methyl, ethyl, n-propyl, isopropyl, n-butyl, isobutyl, tert-butyl, sec-butyl, n-pentyl, 1,1-dimethyl propyl, 1,2-dimethyl propyl, 2,2-dimethyl propyl, 1-ethyl propyl, 2-methyl butyl, 3-methyl butyl, n-hexyl, 1-ethyl-2-methylpropyl, 1,1,2-trimethylpropyl, 1,1-dimethylbutyl, 1,2-dimethylbutyl, 2,2-dimethylbutyl, 1,3-dimethylbutyl, 2-ethylbutyl, 2-methylpentyl, 3-methylpentyl, 4-methylpentyl, 2,3-dimethylbutyl, etc. The alkyl may be substituted or unsubstituted.

“Alkylene” refers to a saturated C₁-C₂₀ straight chain or branched chain aliphatic hydrocarbon group, which has two residues derived from removing two hydrogen atoms from the same carbon atom or two different carbon atoms of maternal alkane, preferably C₁-C₁₀ alkylene, and more preferably C₁-C₆ alkylene. Examples of the alkylene group include, but are not limited to, methylene, 1,1-ethylidene, 1,2-ethylidene, 1,1-propylene, 1,2-propylidene, 1,3-propylidene, 1,4-butylidene, etc. The alkylene can be substituted or unsubstituted.

“Cycloalkyl” refers to a non-aromatic cyclic alkyl group, wherein one or more ring-forming atoms are carbon atoms, including monocyclic rings, polycyclic rings, fused rings, bridged rings and spirocyclic rings, and preferably has a 5-to-7-membered monocyclic ring or a 7-to-10-membered bicyclic or tricyclic ring. Examples of “cycloalkyl” include, but are not limited to, cyclopropyl, cyclopentyl and cyclobutyl. The cycloalkyl can be substituted or unsubstituted.

“Spiroalkyl” refers to a polycyclic group with 5-membered to 18-membered, two or more cyclic structures, and single rings sharing a carbon atom (called a spiro atom) with each other, and each ring comprises one or more double bonds, but none of the rings has a fully conjugated n electron aromatic system, preferably 6-membered to 14-membered, and more preferably 7-membered to 10-membered. The spiroalkyl can be classified into mono-,di- or multi-spiroalkyl according to the number of the sharing spiro atoms between the rings, preferably mono- and di-spiroalkyl, and more preferably 4-membered/5-membered, 4-membered/6-membered, 5-membered/5-membered or 5-membered/6-membered. Non-limiting examples of the “spiroalkyl” include, but are not limited to, spiro[4.5]decyl, spiro[4.4]nonyl, spiro[3.5]nonyl and spiro[2.4]heptyl.

“Fused cycloalkyl” refers to a 5-membered to 18-membered full-carbon polycyclic group with two or more cyclic structures sharing a pair of carbon atoms. One or more rings may comprise one or more double bonds, but none of the rings has a fully conjugated π electron aromatic system, preferably 6-membered to 12-membered, and more preferably 7-membered to 10-membered. According to a number of constituent rings, the fused cycloalkyl can be divided into bicyclic, tricyclic, tetracyclic or polycyclic fused cycloalkyl, preferably bicyclic or tricyclic, and more preferably 5-membered/5-membered or 5-membered/6-membered bicyclic alkyl. Non-limiting examples of the “fused cycloalkyl” include, but are not limited to, bicyclo[3.1.0]hexyl, bicyclo[3.2.0]heptyl-1-alkenyl, bicyclo[3.2.0]heptyl, decalinyl or tetradecahydrophenanthryl.

“Bridged cycloalkyl” refers to a 5-membered to 18-membered full-carbon polycyclic group with two or more cyclic structures sharing a pair of carbon atoms which are not directly connected with each other. One or more rings may comprise one or more double bonds, but none of the ring has a fully conjugated n electron aromatic system, preferably 6-membered to 12-membered, more preferably 7-membered to 10-membered, or preferably 6-membered to 14-membered, more preferably 7-membered to 10-membered. According to the number of constituent rings, the bridged cycloalkyl can be divided into bicyclic, tricyclic, tetracyclic or polycyclic bridged cycloalkyl, preferably bicyclic, tricyclic or tetracyclic, and more preferably bicyclic or tricyclic. Non-limiting examples of the “bridged cycloalkyl” include but are not limited to: (1s,4s)-bicyclo[2.2.1]heptyl, bicyclo[3.2.1]octyl, (1s,5s)-bicyclo[3.3.1]nonyl, bicyclo[2.2.2]octyl, and (1r,5r)-bicyclo[3.3.2]decyl.

“Heterocyclyl”, “heterocyclyl alkyl”, “heterocyclyl” or “heterocyclyl” are used interchangeably in this application, and all refer to non-aromatic heterocyclyl, in which one or more ring-forming atoms are heteroatoms selected from nitrogen, oxygen or S(O)ₜ (wherein t is selected from 0, 1 or 2), including monocyclic, polycyclic, fused rings, bridged rings and spiro rings. The heterocyclyl preferable has a 5-membered to 7-membered monocyclic ring or a 7-membered to 10-membered bicyclic ring or tricyclic ring, which may comprise 1, 2 or 3 atoms selected from nitrogen, oxygen and/or sulfur. Examples of the “heterocyclyl” include, but are not limited to, morpholinyl, oxacyclobutyl, thiomorpholinyl, tetrahydrofurfuryl, tetrahydropyranyl, 1,1-dioxo-thiomorpholinyl, piperidinyl, 2-oxo-piperidinyl, pyrrolidinyl, 2-oxo-pyrrolidinyl, piperazine-2-one, and 8-oxo-3-azido-bicyclo[3.2.1]octyl, piperazinyl, hexahydropyrimidinyl,

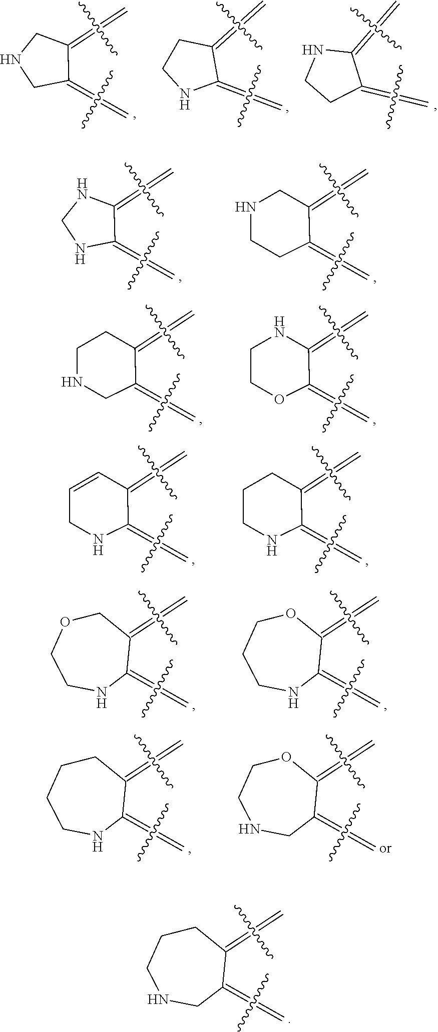

Heterocyclyl can be substituted or unsubstituted.

“Spiroheterocyclyl” refers to a 5-membered to 18-membered polycyclic group with two or more cyclic structures, in which single rings share one atom with each other, and each ring comprises one or more double bonds, but none of the rings has a fully conjugated R electron aromatic system, wherein one or more ring atoms are heteroatoms selected from nitrogen, oxygen or S(O)ₜ (wherein t is selected from 0, 1 or 2), and the rest ring atoms are carbon, preferably 6-membered to 14-membered, and more preferably 7-membered to 10-membered. According to a number of sharing spiro-atoms between the rings, the spiroalkyl can be divided into single-spiroheterocyclyl, double-spiroheterocyclyl or poly-spiroheterocyclyl, preferably single-spiroheterocyclyl and double-spiroheterocyclyl, and more preferably, 4-membered/4-membered, 4-membered/5-membered, 4-membered/6-membered, 5-membered/5-membered or a 5-membered/6-membered single-spiroheterocyclyl. Non-limiting examples of the “spiroheterocyclyl” include, but are not limited to, 1,7-dioxaspiro[4.5]decyl, 2-oxa-7-azaspiro [4.4]nonyl, 7-oxaspiro[3.5]nonyl, 5-oxaspiro[2.4]heptyl,and

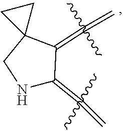 [SMILES: *=C1NCC2(CC2)C1=*]

etc.

“Fused heterocyclyl” refers to a full-carbon polycyclyl with two or more cyclic structures sharing a pair of atoms with each other. One or more rings may comprise one or more double bonds, but none of the rings has a fully conjugated π electron aromatic system, in which one or more ring atoms are heteroatoms selected from nitrogen, oxygen or S(O)ₜ (wherein t is selected from 0, 1 or 2), and the rest ring atoms are carbon, preferably 6-membered to 14-membered, and more preferably 7-membered to 10-membered. According to the number of constituent rings, the fused heterocyclyl can be divided into bicyclic, tricyclic, tetracyclic or polycyclic fused heterocyclyl, preferably bicyclic or tricyclic, and more preferably 5-membered/5-membered or 5-membered/6-membered bicyclic fused heterocyclyl groups. Non-limiting examples of “fused heterocyclyl” include, but are not limited to, octahydropyrrolo[3,4-c]pyrrolyl, octahydro-1H-isoindolyl, 3-azabicyclo[3.1.0]hexyl, and octahydrobenzo[b][1,4]dioxine.

“Bridged heterocyclyl” refers to a 5-membered to 14-membered and 5-membered to 18-membered polycyclyl with two or more cyclic structures sharing two atoms that are not directly connected to each other. One or more rings may comprise one or more double bonds, but none of the rings has a fully conjugated π electron aromatic system, wherein one or more ring atoms are heteroatoms selected from nitrogen, oxygen or S(O)ₜ (wherein t is selected from 0, 1 or 2), and the rest ring atoms are carbon, preferably 6-membered to 14-membered, and more preferably 7-membered to 10-membered. According to the number of constituent rings, the bridged heterocyclyl can be divided into bicyclic, tricyclic, tetracyclic or polycyclic bridged heterocyclyl, preferably bicyclic, tricyclic or tetracyclic, and more preferably bicyclic or tricyclic. Non-limiting examples of the “bridged heterocyclyl” include, but are not limited to, 2-azabicyclo[2.2.1]heptyl, 2-azabicyclo[2.2.2]octyl and 2-azabicyclo[3.3.2]decyl.

“Aryl” refers to a carbocyclic aromatic system including one or two rings, wherein the rings may be connected together in a fused manner. The term “aryl” includes monocyclic or bicyclic aryl, such as aromatic groups of phenyl, naphthyl and tetrahydronaphthyl. The preferred aryl is C₆-C₁₀ aryl, the more preferred aryl is phenyl and naphthyl, and the most preferred aryl is naphthyl. The aryl may be substituted or unsubstituted.

“Heteroaryl” refers to aromatic 5-membered to 6-membered monocyclic ring or 8-membered to 10-membered bicyclic ring, which can comprise 1 to 4 atoms selected from nitrogen, oxygen and/or sulfur. Examples of the “heteroaryl” include but are not limited to furyl, pyridyl, 2-oxo-1,2-dihydropyridyl, pyridazinyl, pyrimidinyl, pyrazinyl, thienyl, isoxazolyl, oxazolyl, oxadiazolyl, imidazolyl, pyrrolyl, pyrazolyl, triazolyl, tetrazolyl, thiazolyl and isothiazolyl, 1,2,3-thiadiazolyl, benzindolizinedioxocyclopentenyl, benzothiophenyl, benzimidazolyl, indolyl, isoindolyl, 1,3-dioxo-isoindolyl, quinolyl, indazolyl, benzisothiazolyl, benzoxazole, benzisothiazolyl, isothiazolyl, 1H-1,2,4-triazolyl, 4H-1,2,4-triazolyl, pyridyl, pyrimidinyl, pyrazine-2(1H)-keto, pyrimidine-4(3H)-keto, pyridazine-3(2H)-keto, 1H-indolyl, 1H-benzo[d]imidazolyl, 1H-pyrido[2,3-c]pyridyl, 3H-imidazo[4,5-c]pyridyl, isoquinoline, quinazolinyl, 2H-isoindolyl, furan[3,2-b]pyridyl, furan[2,3-c]pyridyl, thieno[2,3-c]pyridyl, benzofuryl, benzo[b]thienyl, 1H-pyrido[3,2-b]pyridyl, 2H-pyrido[3,4-c]pyridyl,

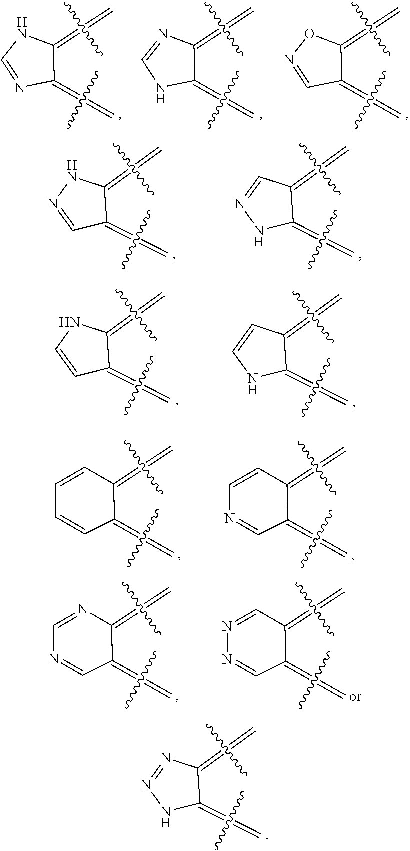

The heteroaryl can be substitute or unsubstituted.

“Alkoxy” refers to a (alkyl-O—) group, wherein the alkyl is as defined herein. C₁-C₆ alkoxy groups are preferred. Examples of alkoxy include, but are not limited to, methoxy, ethoxy, n-propoxy, isopropoxy, n-butoxy, isobutoxy, and tert-butoxy, etc.

“Nitro” refers to a —NO₂ group.

“Hydroxyl” refers to a —OH group.

“Halogen” refers to fluorine, chlorine, bromine and iodine.

“Amino” refers to —NH₂.

“Cyano” refers to —CN.

“Benzyl” refers to —CH₂-phenyl.

“Carboxyl” refers to —C(O)OH.

“Carboxylic ester” refers to —C(O)O-alkyl or —C(O)O-cycloalkyl, wherein the alkyl and cycloalkyl are defined as above.

“Hydroxyalkyl” refers to hydroxyl-substituted alkyl, wherein the alkyl is as defined above.

“Aminoalkyl” refers to amino-substituted alkyl, wherein the alkyl is as defined above.

“Haloalkyl” refers to halogen-substituted alkyl, wherein the alkyl is as defined above.

“Haloalkoxy” refers to halogen-substituted alkoxy, wherein the alkoxy is as defined above.

“DMSO” refers to dimethyl sulfoxide.

“BOC” refers to tert-butoxycarbonyl.

“Bn” refers to benzyl.

“THP” refers to 2-tetrahydropyranyl.

“TFA” refers to trifluoroacetic acid.

“Ts” refers to tosyl.

“Leaving group”, or leaving group, is an atom or functional group detached from a larger molecule in a chemical reaction, which is a term used in nucleophilic substitution reaction and elimination reaction. In nucleophilic substitution reaction, the reactant attacked by nucleophiles is called substrate, and the atom or atomic group that breaks off from a substrate molecule with a pair of electrons is called a leaving group. The groups that accept electrons easily and bear negative charges strongly are good leaving groups. When the pKa of the conjugated acid of the leaving group is smaller, the leaving group is easier to be separated from other molecules. The reason is that when the pKa of the conjugated acid of the leaving group is smaller, the corresponding leaving group does not need to combine with other atoms, and the trend of existence in the form of anion (or electrically neutral leaving group) is also enhanced. The common leaving groups include but are not limited to halogen, methanesulfonyl, —OTs or —OH.

“Substituted” refers to that one or more hydrogen atoms in a group, preferably at most five, more preferably 1-3 hydrogen atoms, are independently replaced by a corresponding number of substituents. It goes without saying that the substituents are only in their possible chemical positions, and those skilled in the art may determine (by experiment or theory) possible or impossible substitutions without excessive efforts. For example, amino groups or hydroxyl groups with free hydrogen may be unstable when combined with carbon atoms with unsaturated (such as olefinic) bonds.

“Substituted” or “substituted” in the description, unless otherwise specified, means that the group may be substituted by one or more substitutents selected from the following groups: alkyl, alkenyl, alkynyl, alkoxy, alkylthio, alkylamino, halogen, sulfanyl, hydroxyl, nitro, cyano, cycloalkyl, heterocyclyl, aryl, heteroaryl, cycloalkoxy, heterocycloalkoxy, cycloalkthio, heterocycloalkthio, amino, haloalkyl, hydroxyalkyl, carboxyl, carboxylic acid ester, ═O, —OR⁶, —C(O)R⁶, —C(O)OR⁶, —NHC(O)R⁶, —NHC(O)OR⁶, —NR⁷R⁸, —C(O)NR⁷R⁸, —CH₂NHC(O)OR⁶, —CH₂NR⁷R⁸ or —S(O)ᵣR⁶.

R⁶ is selected from hydrogen atom, alkyl, cycloalkyl, heterocyclyl, aryl or heteroaryl; wherein alkyl, cycloalkyl, heterocyclyl, aryl or heteroaryl is optionally further substituted by one or more substituents selected from hydroxyl, halogen, nitro, cyano, alkyl, alkoxy, haloalkyl, haloalkoxy, cycloalkyl, heterocyclyl, aryl, heteroaryl, ═O, —C(O)R⁹, —C(O)OR⁹, —OC(O)R⁹, —NR¹⁰R¹¹, —C(O)NR¹⁰R¹¹, —SO₂NR¹⁰R¹¹ or —NR¹⁰C(O)R¹¹.

R⁷ and R⁸ are each independently selected from hydrogen atom, hydroxyl, halogen, alkyl, alkoxy, cycloalkyl, heterocyclyl, aryl or heteroaryl, wherein alkyl, alkoxy, cycloalkyl, heterocyclyl, aryl or heteroaryl is optionally further substituted by one or more substituents selected from hydroxyl, halogen, nitro, cyano, alkyl, alkoxy, cycloalkyl, heterocyclyl, aryl, heteroaryl, ═O, —C(O)R⁹, —C(O)OR⁹, —OC(O)R⁹, —NR¹⁰R¹¹, —C(O)NR¹⁰R¹¹, —SO₂NR¹⁰R¹¹ or —NR¹⁰C(O)R¹¹;

or, R⁷ and R⁸ and the atoms attached thereto form a 4-8-membered heterocyclyl, wherein the 4-8-membered heterocyclyl comprises one or more N, O or S(O)r, and the 4-8-membered heterocyclyl group is optionally further substituted by one or more substituents selected from hydroxyl, halogen, nitro, cyano, alkyl, alkoxy, cycloalkyl, heterocyclyl, aryl, heteroaryl, ═O, —C(O)R⁹, —C(O)OR⁹, —OC(O)R⁹, —NR¹⁰R¹¹, —C(O)NR¹⁰R¹¹, —SO₂NR¹⁰R¹¹ or —NR¹⁰C(O)R¹¹.

R⁹, R¹⁰ and R¹¹ are each independently selected from hydrogen atom, alkyl, amino, cycloalkyl, heterocyclyl, aryl or heteroaryl, wherein the alkyl, cycloalkyl, heterocyclyl, aryl or heteroaryl is optionally further substituted by one or more substituents selected from hydroxyl, halogen, nitro, amino, cyano, alkyl, alkoxy, cycloalkyl, heterocyclyl, aryl, heteroaryl, carboxyl or carboxylate.

r is selected from 0, 1 or 2;

The compound of the present invention can comprise an asymmetric center or a chiral center, and therefore exist in different stereoisomeric forms. It is expected that all stereoisomeric forms of the compounds of the present invention, including but not limited to diastereomers, enantiomers, atropisomers and geometric (conformational) isomers and mixtures thereof, such as racemic mixtures, are all within the scope of the present invention.

Unless otherwise specified, the structure described in the present invention also includes all isomers of the structure (for example, diastereomers, enantiomers, atropisomers and geometric (conformational) isomer forms; for example, R and S configurations of each asymmetric center, (Z) and (E) double bond isomers, and (Z) and (E) conformational isomers. Therefore, the single stereoisomer, an enantiomeric mixture, a diastereomeric mixture and a geometric (conformational) isomer mixture of the compound of the present invention are all within the scope of the present invention.

“Pharmaceutically acceptable salts” refers to some salts of the above compounds that can maintain original biological activities thereof and are suitable for medical use. The pharmaceutically acceptable salt of the compound as shown by general formula (I) can be a metal salt or an amine salt formed with a suitable acid.

A “pharmaceutical composition” refers to a mixture including one or more compounds described herein or physiologically acceptable salts thereof or a prodrug thereof and other chemical components, as well as other components such as physiologically acceptable carriers and excipients. The purpose of the pharmaceutical composition is to promote the drug administration to organisms, facilitate the absorption of active ingredients and further exert biological activity.

### Synthesis Method of the Compound of the Present Invention

In order to complete the purpose of the present invention, the present invention adopts the following technical solution:

The present invention provides a preparation method of a compound or a stereoisomer, a tautomer as shown by general formula (I) or a pharmaceutically acceptable salt thereof, which comprises the following steps:

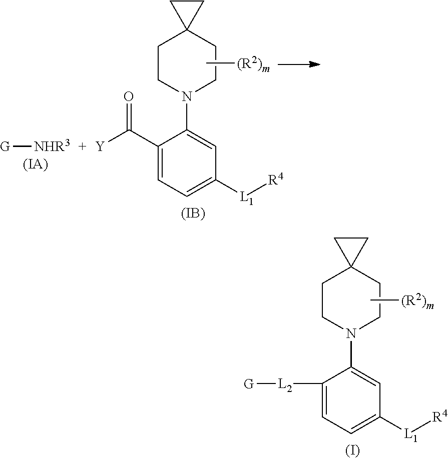

A condensation reaction was carried out between the compound as shown by general formula (IA) and the compound as shown by general formula (IB), and optionally further substitution reaction was carried out to obtain the compound as shown by general formula (I),

Wherein,
L₂ is selected from

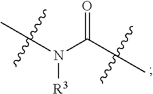

Y is selected from hydroxyl or chlorine; and
G, L₁, R²-R⁴ and m are defined as in general formula (I).

### Embodiment

The present invention is further described in conjunction with the following examples, but these examples do not limit the scope of the present invention.

### EXAMPLES

The preparation of representative compounds as shown by general formula (I) and related structural identification data are given in the examples. It should be noted that the following examples are used to illustrate the present invention and not to limit the present invention. A ¹H NMR spectrum was measured by a Bruker instrument (400 MHz), and a chemical shift was expressed in ppm. An internal standard of tetramethylsilane (0.00 ppm) was used. A ¹H NMR representation method is that: s=single peak, d=double peak, t=triple peak, m=multiple peak, br=broadened, dd=double peak of double peak, dt=double peak of triple peak. If a coupling constant was provided, a unit thereof is Hz.

Mass spectrometry was determined by a LC/MS instrument, and an ionization mode could be ESI or APCI.

Yantai Huanghai HSGF254 or Qingdao GF254 silica gel plates were used as the silica gel plates for a thin-layer chromatography, and the specifications of the silica gel plates used for a thin-layer chromatography (TLC) were 0.15 mm-0.2 mm, and the specifications of the products separated and purified by the TLC were 0.4 mm-0.5 mm.

Yantai Huanghai silica gel 200-300-mesh silica gel was generally used for a column chromatography as a carrier.

In the following examples, unless otherwise specified, all temperatures were Celsius. Unless otherwise specified, various starting materials and reagents were commercially available or synthesized according to known methods, and the commercially available raw materials and reagents were directly used without further purification. Unless otherwise specified, commercially available manufacturers included but were not limited to Aldrich Chemical Company, ABCR GmbH & Co.KG, Acros Organics, Guangzan Chemical Technology Co., Ltd. and Jingyan Chemical Technology Co., Ltd.

CD₃OD: deuterated methanol.

CDCl₃: deuterated chloroform.

DMSO-d₆: deuterated dimethyl sulfoxide.

An argon atmosphere means that a reaction bottle is connected with an argon balloon with a volume of about 1 L.

Unless otherwise specified in the examples, a solution in the reaction referred to an aqueous solution.

The compound was purified by a silica gel column chromatography and a reversed-phase column chromatography, wherein an eluent system was selected from: A: petroleum ether and ethyl acetate system; B: dichloromethane and methanol system; C: dichloromethane:ethyl acetate; D: trifluoroacetic acid aqueous solution and acetonitrile system. A volume ratio of the solvent was different according to the polarity of the compound, and could also be adjusted by adding a small amount of acidic or alkaline reagents, such as acetic acid or triethylamine.

### Example 1

### N-(3-(4,4-difluoropiperidin-1-yl)-2-methoxyphenyl)-4-((2-hydroxyethyl)sulfonamido)-2-(6-azasp iro[2.5]octan-6-yl)benzamide

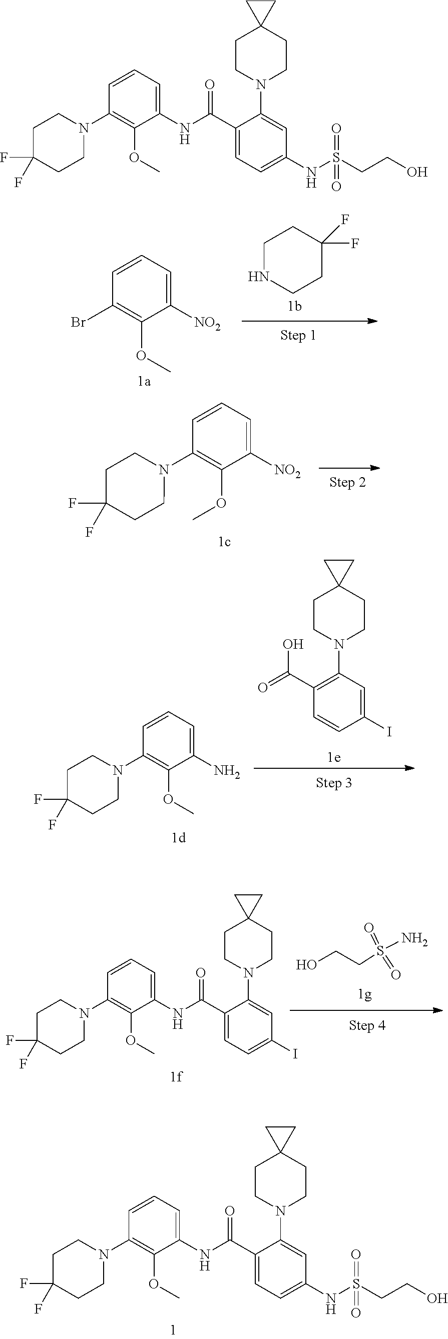

### Step 1

### 4,4-difluoro-1-(2-methoxy-3-nitrophenyl)piperidine

1-bromo-2-methoxy-3-nitro-benzene 1a (500 mg, 2.15 mmol, commercially available), 4,4-difluoropyridine 1b (313.22 mg, 2.59 mmol, commercially available), palladium acetate (48.38 mg, 215.49 μmol), 4,5-bisdiphenylphosphino-9,9-dimethylxanthene (249.37 mg, 430.98 μmol), and cesium carbonate (2.11 g, 6.46 mmol) were dissolved in 1,4-dioxane (5 mL) and the mixture was subjected to argon sparging for 15 minutes, then heated to 100° C. and stirred for 16 hours. Water (20 mL) was added, and the mixture was extracted with ethyl acetate (30 mL×2). The organic phases were combined, and washed with saturated sodium chloride (20 mL), dried over anhydrous sodium sulfate, filtered and concentrated. The residue was purified by silica gel chromatography (eluent: system A) to obtain 4,4-difluoro-1-(2-methoxy-3-nitrophenyl)piperidine 1c (400 mg) with a yield of 68.18%.

MS m/z (ESI): 273.1 [M+1]

### Step 2

### 3-(4,4-difluoropiperidin-1-yl)-2-methoxyaniline

4,4-difluoro-1-(2-methoxy-3-nitrophenyl)piperidine 1c (400 mg, 1.47 mmol) was added into methanol (2 mL), then 10% palladium carbon (31.27 mg, 293.85 mol) was added. The mixture was subjected to hydrogen purging for three times, and then reacted at room temperature for 3 hours. The reaction mixture was filtered and concentrated under a reduced pressure. The residue was purified by silica gel chromatography (eluent: system A) to obtain 3-(4,4-difluoropiperidin-1-yl)-2-methoxyaniline 1d (170 mg) with a yield of 47.76%.

MS m/z (ESI): 243.1 [M+1]

### Step 3

### N-(3-(4,4-difluoropiperidin-1-yl)-2-methoxyphenyl)-4-iodo-2-(6-azaspiro[2.5]octan-6-yl)benzam ide

4-iodo-2-(6-azaspiro[2.5]octan-6-yl)-benzoic acid 1e (265.39 mg, 742.99 μmol, self-prepared according to the method in published patent WO2020132648) and 3-(4,4-difluoropiperidin-1-yl)-2-methoxyphenyl 1d (150 mg, 619.16 gmol) were added into acetonitrile (1.5 mL), then N-methylimidazole (254.16 mg, 3.10 mmol) was added, and the mixture was stirred for 5 minutes. Then N,N,N′,N′-tetramethylchloroformamidine hexafluorophosphate (868.59 mg, 3.10 mmol) was added to react for 3 hours. Water (20 mL) was added, and the obtained mixture was extracted with ethyl acetate (30 mL×2). The organic phases were combined, washed with saturated sodium chloride (10 mL), dried over anhydrous sodium sulfate, filtered and concentrated. The residue was purified by silica column chromatography (eluent: system A) to obtain N-(3-(4,4-difluoropiperidin-1-yl)-2-methoxyphenyl)-4-iodo-2-(6-azaspiro[2.5]octan-6-yl)benzam ide if (250 mg) with a yield of 46.53%.

MS m/z (ESI): 582.2 [M+1]

### Step 4

### N-(3-(4,4-difluoropiperidin-1-yl)-2-methoxyphenyl)-4-((2-hydroxyethyl)sulfonamido)-2-(6-azaspiro[2.5]octan-6-yl)benzamide

2-hydroxyethane-1-sulfonamide 1g (10.76 mg, 85.99 μmol) and N-(3-(4,4-difluoropiperidin-1-yl)-2-methoxyphenyl)-4-iodo-2-(6-azaspiro[2.5]octan-6-yl)benzam ide if (25 mg, 43.00 μmol) were added into N,N-dimethylformamide(1 mL), then 2-(methylamino)acetic acid (3.83 mg, 43.00 μmol), tripotassium phosphate trihydrate (57.25 mg, 214.99 μmol) and cuprous iodide (4.77 mg, 21.50 mol) were added and the obtained mixture was heated to 110° C. for 3 hours under argon protection. After cooling, ethyl acetate (20 mL) and water (10 mL) were added to the reaction mixture for extraction, the organic phases were concentrated. The residue was purified by preparative liquid chromatography (separation column AKZONOBEL Kromasil; 250×21.2 mm I.D.; 5 μm, 20 mL/min; a mobile phase A: 0.05% TFA+H₂O, and a mobile phase B: CH₃CN) to obtain N-(3-(4,4-difluoropiperidin-1-yl)-2-methoxyphenyl)-4-((2-hydroxyethyl)sulfonamido)-2-(6-azasp iro[2.5]octan-6-yl)benzamide 1 (3 mg) with a yield of 11.45%.

MS m/z (ESI): 579.2 [M+1]

¹H NMR (400 MHz, DMSO-d₆) δ 11.64 (s, 1H), 8.17 (d, J=8.2 Hz, 1H), 7.91 (d, J=8.5 Hz, 1H), 7.18 (s, 1H), 7.04 (d, J=7.9 Hz, 2H), 6.77 (d, J=8.1 Hz, 1H), 3.81 (s, 3H), 3.77 (t, J=6.5 Hz, 2H), 3.39 (s, 2H), 3.18 (t, J=5.5 Hz, 4H), 2.96 (t, J=5.2 Hz, 4H), 2.24-2.08 (m, 4H), 1.53 (s, 4H), 0.31 (s, 4H).

### Example 2

### N-(2-cyano-3-(4,4-difluoropiperidin-1-yl)phenyl)-4-((2-hydroxyethyl)sulfonamido)-2-(6-azaspiro [2.5]octan-6-yl)benzamide

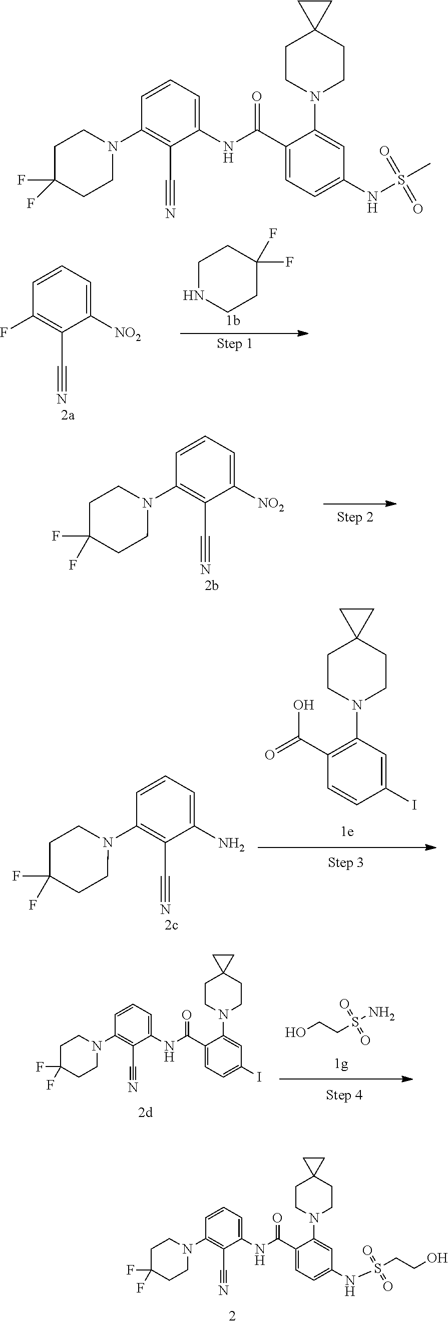

### Step 1

### 2-(4,4-difluoropiperidin-1-yl)-6-nitrobenzonitrile

2-fluoro-6-nitrobenzonitrile 2a (400 mg, 2.41 mmol, commercially available), 4,4-difluorpiperidine 1b (437.52 mg, 3.61 mmol) and potassium carbonate (665.61 mg, 4.82 mmol) were dissolved in N,N-dimethylformamide (2 mL), and the mixture was heated to 80° C., and stirred for 4 hours. Water (20 mL) was added, and the mixture was extracted with ethyl acetate (30 mL×2). The organic phases were combined, washed with saturated sodium chloride (20 mL), dried over anhydrous sodium sulfate, filtered, and concentrated. The residue was dissolved in ethyl acetate, petroleum ether was added, and a solid was precipitated to obtain 2-(4,4-difluoropiperidin-1-yl)-6-nitrobenzonitrile 2b (500 mg) with a yield of 77.70%.

MS m/z (ESI): 268.1 [M+1]

### Step 2

### 2-amino-6-(4,4-difluoropiperidin-1-yl)benzonitrile

2-(4,4-difluoropiperidin-1-yl)-6-nitrobenzonitrile 2b (500 mg, 1.87 mmol) was dissolved in methanol (3 mL), 10% palladium carbon (50 mg, 374.21 mol) was added, and the mixture was subjected to hydrogen purging for three times and stirred at room temperature for 6 hours. The reaction mixture was filtered and concentrated under a reduced pressure. The residue was purified by silica gel chromatography (eluent: system A) to obtain 2-amino-6-(4,4-difluoropiperidin-1-yl)benzonitrile 2c (300 mg) with a yield of 67.58%.

MS m/z (ESI): 238.2 [M+1]

¹H NMR (400 MHz, Chloroform-d) δ 7.21 (t, J=8.1 Hz, 1H), 6.43-6.32 (m, 2H), 4.23 (d, J=80.5 Hz, 2H), 3.32-3.24 (m, 4H), 2.20 (tt, J=13.7, 5.7 Hz, 4H).

### Step 3

### N-(2-cyano-3-(4,4-difluoropiperidin-1-yl)phenyl)-4-iodo-2-(6-azaspiro[2.5]octan-6-yl)benzamide

4-iodo-2-(6-azaspiro[2.5]octan-6-yl)benzoic acid 1e (248.41 mg, 695.47 μmol) and 2-amino-6-(4,4-difluoropiperidin-1-yl)benzonitrile 2c (150 mg, 632.25 μmol) were added into 1,4-dioxane (1 mL), then N,N,N′,N′-tetramethylchloroformamidine hexafluorophosphate (532.19 mg, 1.90 mmol) and N-methylimidazole (155.72 mg, 1.90 mmol) were added, and the mixture was heated to 100° C. to react for 3 hours. Water (20 mL) was added to the mixture, and the obtained mixture was extracted with ethyl acetate (30 mL×2). The organic phases were combined, washed with saturated sodium chloride (10 mL), dried over anhydrous sodium sulfate, filtered and concentrated. The residue was purified by silica column chromatography (eluent: system A) to obtain N-(2-cyano-3-(4,4-difluoropiperidin-1-yl)phenyl)-4-iodo-2-(6-azaspiro[2.5]octan-6-yl)benzamide 2d (340 mg) with a yield of 93.29%.

MS m/z (ESI): 557.1 [M+1]

### Step 4

### N-(2-cyano-3-(4,4-difluoropiperidin-1-yl)phenyl)-4-((2-hydroxyethyl)sulfonamido)-2-(6-azaspiro [2.5]octan-6-yl)benzamide

2-hydroxyethane-1-sulfonamide1g (147.64 mg, 1.18 mmol) and N-(2-cyano-3-(4,4-difluoropiperidin-1-yl)phenyl)-4-iodo-2-(6-azaspiro[2.5]octan-6-yl)benzamide 2d (340 mg, 589.85 μmol) were dissolved in N,N-dimethylformamide(5 mL), then 2-(methylamino)acetic acid (52.55 mg, 589.85 μmol), tripotassium phosphate trihydrate (785.42 mg, 2.95 mmol)) and cuprous iodide (65.49 mg, 294.92 μmol) were added. The mixture was subjected to sparging with argon for 15 minutes, heated to 100° C. and stirred for 10 hours. After cooling, ethyl acetate (20 mL) and water (10 mL) were added to the reaction mixture for extraction, and organic phases were concentrated. The residue was purified by preparative liquid chromatography (separation column AKZONOBEL Kromasil; 250×21.2 mm I.D.; 5 μm, 20 mL/min; a mobile phase A: 0.05% NH₄HCO₃+H₂O, and a mobile phase B: CH₃CN) to obtain N-(2-cyano-3-(4,4-difluoropiperidin-1-yl)phenyl)-4-((2-hydroxyethyl)sulfonamido)-2-(6-azaspiro [2.5]octan-6-yl)benzamide 2 (76 mg) with a yield of 21.88%.

MS m/z (ESI): 574.3 [M+1]

¹H NMR (400 MHz, DMSO-d₆) δ 11.68 (s, 1H), 7.86 (d, J=8.5 Hz, 1H), 7.75 (d, J=8.3 Hz, 1H), 7.60 (t, J=8.3 Hz, 1H), 7.17 (s, 1H), 7.02 (d, J=8.3 Hz, 2H), 3.75 (t, J=6.5 Hz, 2H), 3.31 (s, 2H), 3.28 (t, J=6.0 Hz, 4H), 3.00 (t, J=5.4 Hz, 4H), 2.18 (dq, J=16.0, 9.7, 7.3 Hz, 4H), 1.53 (s, 4H), 0.33 (s, 4H).

### Example 3

### N-(6-(4,4-difluoropiperidin-1-yl)pyridazin-4-yl)-4-((2-hydroxyethyl)sulfonamido)-2-(6-azaspiro[2.5]octan-6-yl)benzamide

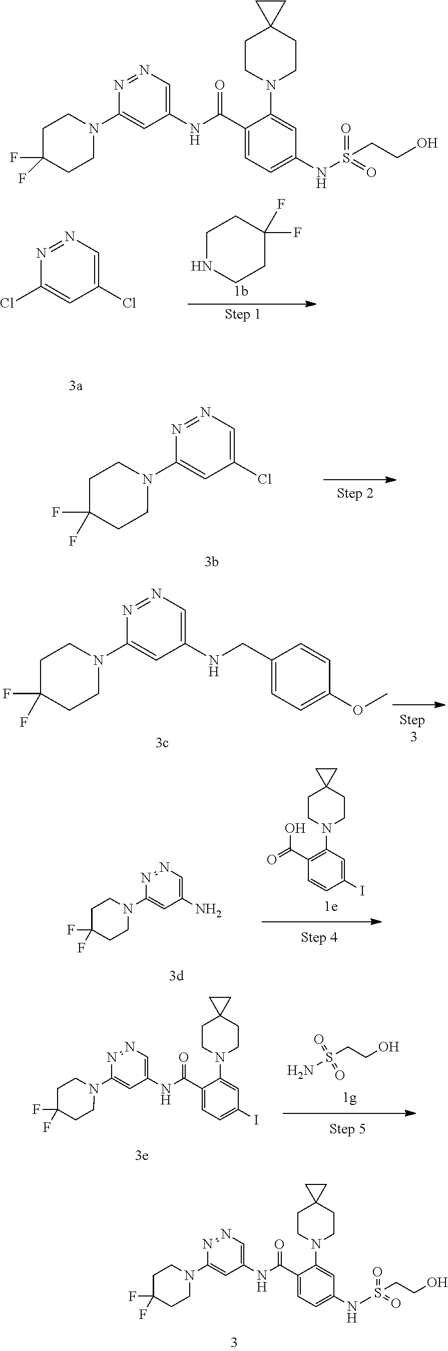

### Step 1

### 5-chloro-3-(4,4-difluoropiperidin-1-yl)pyridazine

3,5-dichloropyridazine 3a (500 mg, 3.36 mmol) and 4,4-difluoropiperidin 1b (447.18 mg, 3.69 mmol) were dissolved in methanol (5 mL), heated to 60° C. and stirred for 18 hours. Water (20 mL) was added, and the mixture was extracted with ethyl acetate (30 mL×2). The organic phases were combined, washed with saturated sodium chloride (20 mL), dried over anhydrous sodium sulfate, filtered and concentrated. The residue was purified by silica gel column chromatography (eluent: system A) to obtain 5-chloro-3-(4,4-difluoropiperidin-1-yl)pyridazine 3b (400 mg) with a yield of 51.01%.

MS m/z (ESI): 234.1 [M+1]

### Step 2

### 6-(4,4-difluoropiperidin-1-yl)-N-(4-methoxybenzyl)pyridazin-4-amine

5-chloro-3-(4,4-difluoropiperidin-1-yl)pyridazine 3b (400 mg, 1.71 mmol), (4-methoxyphenyl) methylamine (281.82 mg, 2.05 mmol), methanesulfonic acid (2-dicyclohexylphosphine)-3,6-dimethoxy-2′,4′,6′-triisopropyl-1,1′-biphenyl)(2′-amino-1,1′-biphe nyl-2-yl)palladium (II) (155.37 mg, 171.20 μmol) and sodium tert-butoxide (493.58 mg, 5.14 mmol) were dissolved in 1,4-dioxane (5 mL), then the mixture was subjected to argon purging for three times, heated to 80° C. and stirred for 10 hours. After concentration, the residue was purified by silica gel column chromatography (eluent: system B) to obtain 6-(4,4-difluoropiperidin-1-yl)-N-(4-methoxybenzyl)pyridazin-4-amine 3c (500 mg) with a yield of 87.35%.

MS m/z (ESI): 335.1 [M+1]

### Step 3

### 6-(4,4-difluoropiperidin-1-yl)pyridazin-4-amine

(6-(4,4-difluoropiperidin-1-yl)-N-(4-methoxybenzyl)pyridazin-4-amine 3c (500 mg, 1.50 mmol) was dissolved in trifluoromethane (2 mL), and stirred at room temperature overnight. The reaction mixture was concentrated under reduced pressure to obtain 6-(4,4-difluoropiperidin-1-yl)pyridazin-4-amine 3d (450 mg) with a yield of 91.68%.

MS m/z (ESI): 215.2 [M+1]

### Step 4

### N-(6-(4,4-difluoropiperidin-1-yl)pyridazin-4-yl)-4-iodo-2-(6-azaspiro[2.5]octan-6-yl)benzamide

6-(4,4-difluoropiperidin-1-yl)pyridazin-4-amine 3d (400 mg, 1.22 mmol) was dissolved in acetonitrile (151.95 μL), then N,N-diethylethylamine (616.56 mg, 6.09 mmol, 846.93 μL) and 4-iodo-2-(6-azaspiro[2.5]octan-6-yl) benzoic acid 1e (478.80 mg, 1.34 mmol) were added dropwise, and N,N,N′,N′-tetramethylchloroformamidine hexafluorophosphate (1.03 g, 3.66 mmol) was added, and the mixture was heated to 60° C. and stirred for 1 hour. Water (20 mL) was added, the mixture was extracted with ethyl acetate (30 mL×2). The organic phases were combined, washed with saturated sodium chloride (10 mL), dried over anhydrous sodium sulfate, filtered and concentrated. The residue was purified by silica gel column chromatography (eluent: system A) to obtain N-(6-(4,4-difluoropiperidin-1-yl)pyridazin-4-yl)-4-iodo-2-(6-azaspiro[2.5]octan-6-yl)benzamide 3e (300 mg) with a yield of 44.49%.

MS m/z (ESI): 554.1 [M+1]

### Step 5

### N-(6-(4,4-difluoropiperidin-1-yl)pyridazin-4-yl)-4-((2-hydroxyethyl)sulfonamido)-2-(6-azaspiro[ 2.5]octan-6-yl)benzamide

2-hydroxyethane-1-sulfonamide 1g (135.69 mg, 1.08 mmol) and N-(6-(4,4-difluoropiperidin-1-yl)pyridazin-4-yl)-4-iodo-2-(6-azaspiro[2.5]octan-6-yl)benzamide 3e (300 mg, 542.12 μmol) were dissolved in N,N-dimethylformamide(5 mL), then tripotassium phosphate trihydrate (721.86 mg, 2.71 mmol), 2-(methylamino)acetic acid (48.30 mg, 542.12 mol) and cuprous iodide(60.19 mg, 271.06 μmol) were added and heated to 110° C. to react for 3 hours under argon protection. Water (20 mL) was added, the obtained mixture was extracted with ethyl acetate (30 mL×2). The organic phases were combined, washed with saturated sodium chloride (10 mL), dried over anhydrous sodium sulfate, filtered and concentrated. The residue was purified by preparative liquid chromatography (separation column AKZONOBEL Kromasil; 250×21.2 mm I.D.; 5 μm, 20 mL/min; a mobile phase A: 0.05% NH₄HCO₃+H₂O, and a mobile phase B: CH₃CN) to obtain N-(6-(4,4-difluoropiperidin-1-yl)pyridazin-4-yl)-4-((2-hydroxyethyl)sulfonamido)-2-(6-azaspiro[2.5]octan-6-yl)benzamide 3 (21 mg) with a yield of 6.96%.

MS m/z (ESI): 551.2 [M+1]

¹H NMR (400 MHz, DMSO-d₆) δ 13.06 (s, 1H), 8.86 (d, J=2.8 Hz, 1H), 8.04 (d, J=8.6 Hz, 1H), 7.97 (d, J=2.8 Hz, 1H), 7.28 (s, 1H), 7.12 (d, J=8.7 Hz, 1H), 3.76 (t, J=6.5 Hz, 2H), 3.61 (t, J=5.8 Hz, 4H), 3.38 (s, 2H), 2.99 (t, J=5.3 Hz, 4H), 2.09 (tt, J=13.8, 5.3 Hz, 4H), 1.69 (s, 4H), 0.40 (s, 4H).

### Example 4

### N-(1-(4,4-difluorocyclohexyl)-6-oxo-1,6-dihydropyridin-3-yl)-4-((2-hydroxyethyl)sulfonamido)-2-(6-azaspiro[2.5]octan-6-yl)benzamide

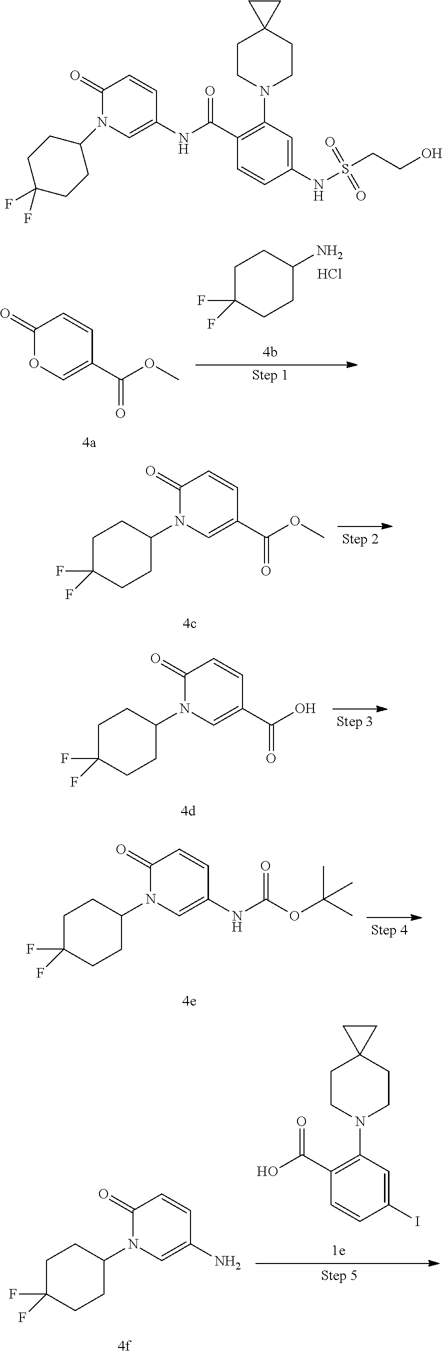

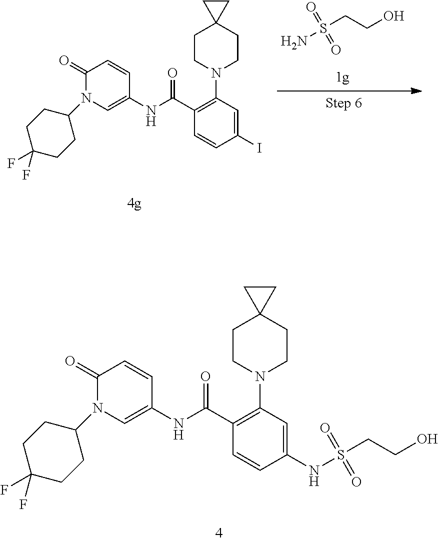 [SMILES: O=C(Nc1ccc(=O)n(C2CCC(F)(F)CC2)c1)c1ccc(I)cc1N1CCC2(CC1)CC2 ; NS(=O)(=O)CCO ; O=C(Nc1ccc(=O)n(C2CCC(F)(F)CC2)c1)c1ccc(NS(=O)(=O)CCO)cc1N1CCC2(CC1)CC2]

### Step 1

### Methyl 1-(4,4-difluorocyclohexyl)-6-oxo-1,6-dihydropyridine-3-carboxylate

4,4-difluorocyclohexan-1-amine hydrochloride 4b (1.04 g, 6.07 mmol, commercially available), methyl 2-oxo-2H-pyran-5-carboxylate 4a (850 mg, 5.52 mmol) and triethylamine (1.12 g, 11.03 mmol) were added into methanol (20 mL), and the reaction mixture was heated to reflux for 4 hours. After vacuum concentration, methyl 1-(4,4-difluorocyclohexyl)-6-oxo-1,6-dihydropyridine-3-carboxylate 4c (1.4 g) was obtained as a crude product which was directly used in the next step with a yield of 93%.

MS m/z (ESI): 272.1 [M+1]

### Step 2

### 1-(4,4-difluorocyclohexyl)-6-oxo-1,6-dihydropyridine-3-carboxylic acid

An aqueous solution (10 mL) of sodium hydroxide (619.29 mg, 15.48 mmol) was added into methyl 1-(4,4-difluorocyclohexyl)-6-oxo-1,6-dihydropyridine-3-methyl carboxylate 4c (1.4 g, 5.16 mmol) in a mixed solvent of methanol and tetrahydrofuran (10 mL), and the mixture was stirred at room temperature for 0.5 hours. Hydrochloric acid (1M) was added to the system to adjust a pH to 3-4, then ethyl acetate (30 mL) was added, the mixture was extracted. The water phase was further extracted with ethyl acetate (30 mL×2), and the organic phases were combined, washed with saturated sodium chloride solution (30 mL), dried over anhydrous sodium sulfate, and concentrated under reduced pressure to obtain 1-(4,4-difluorocyclohexyl)-6-oxo-1,6-dihydropyridine-3-carboxylic acid 4d (1.1 g) with a yield of 82.86%.

MS m/z (ESI): 258.1 [M+1]

### Step 3

### Tert-butyl (1-(4,4-difluorocyclohexyl)-6-oxo-1,6-dihydropyridin-3-yl)carbamate

1-(4,4-difluorocyclohexyl)-6-oxo-1,6-dihydropyridin-3-carboxylic acid 4d (550 mg, 2.14 mmol), diphenyl azide phosphate (647.26 mg, 2.35 mmol) and triethylamine (324.54 mg, 3.21 mmol) were added into tert-butanol (1 mL), and the mixture was heated to 80° C. and stirred for 16 hours. The reaction mixture was concentrated under reduced pressure, and the residue was purified by silica gel column chromatography (eluent: system A) to obtain tert-butyl (1-(4,4-difluorocyclohexyl)-6-oxo-1,6-dihydropyridin-3-yl)carbamate 4e (450 mg) with a yield of 64.10%.

MS m/z (ESI): 329.1 [M+1]

### Step 4

### 5-Amino-1-(4,4-difluorocyclohexyl)pyridin-2(1H)-one

Tert-butyl (1-(4,4-difluorocyclohexyl)-6-oxo-1,6-dihydropyridin-3-yl)carbamate 4e (450 mg, 1.37 mmol) was added into hydrogen chloride in ethyl acetate (4 M, 1 mL) and stirred at room temperature for 1 hour. The reaction mixture was concentrated under reduced pressure to obtain 5-amino-1-(4,4-difluorocyclohexyl)pyridin-2(1H)-one 4f (361 mg) as a crude product which was directly used in the next step with a yield of 99.51%.

MS m/z (ESI): 229.1 [M+1]

### Step 5

### N-(1-(4,4-difluorocyclohexyl)-6-oxo-1,6-dihydropyridin-3-yl)-4-iodo-2-(6-azaspiro[2.5]octan-6-yl)benzamide

5-Amino-1-(4,4-difluorocyclohexyl)pyridin-2(1H)-one 4f (88.93 mg, 335.96 μmol), 4-iodo-2-(6-azaspiro[2.5]octan-6-yl) benzoic acid 1e (100 mg, 279.97 μmol), N-methylimidazole (69.80 mg, 839.90 gmol) and triethylamine (84.99 mg, 839.90 gmol) were added into acetonitrile (1 mL), and the mixture was stirred at room temperature for 10 minutes, then N,N,N′,N′-tetramethylchloroformamidine hexafluorophosphate (235.66 mg, 839.90 μmol) was added and the mixture was stirred at room temperature for 4 hours. The reaction mixture was concentrated under reduced pressure, the residue was purified by silica gel column chromatography (eluent: system A) to obtain N-(1-(4,4-difluorocyclohexyl)-6-oxo-1,6-dihydropyridin-3-yl)-4-iodo-2-(6-azaspiro[2.5]octan-6-yl)benzamide 4g (78 mg), with a yield of 49.10%.

MS m/z (ESI): 568.2 [M+1]

### Step 6

### N-(1-(4,4-difluorocyclohexyl)-6-oxo-1,6-dihydropyridin-3-yl)-4-((2-hydroxyethyl)sulfonamido)- 2-(6-azaspiro[2.5]octan-6-yl)benzamide

N-(1-(4,4-difluorocyclohexyl)-6-oxo-1,6-dihydropyridin-3-yl)-4-iodo-2-(6-azaspiro[2.5]octa n-6-yl)benzamide 4g (20 mg, 35.25 mol), 2-hydroxyethne-1-sulfonamide 1g (4.85 mg, 38.77 μmol), 2-(methylamino)acetic acid (4.71 mg, 52.87 μmol), cuprous iodide (2.01 mg, 10.57 μmol) and tripotassium phosphate trihydrate (46.93 mg, 176.24 μmol) were added into N,N-dimethylformamide (1 mL), and the mixture was heated to 110° C. and stirred for 6 hours after argon purging. The reaction mixture was filtered and concentrated, and the residue was purified by preparative liquid chromatography (separation column AKZONOBEL Kromasil; 250×21.2 mm I.D.; 5 μm, 20 mL/min; a mobile phase A: 0.05% TFA+H₂O, and a mobile phase B: CH₃CN) to obtain N-(1-(4,4-difluorocyclohexyl)-6-oxo-1,6-dihydropyridin-3-yl)-4-((2-hydroxyethyl)sulfonamido)-2-(6-azaspiro[2.5]octan-6-yl)benzamide 4 (1.67 mg), with a yield of 8.15%.

MS m/z (ESI): 565.2 [M+1]

¹HNMR (400 MHz, DMSO-d₆) δ 11.18 (s, 1H), 10.08 (s, 1H), 8.39 (d, J=8.0 Hz, 1H), 7.75 (d, J=8.0 Hz, 1H), 7.57 (dd, J=4.0 Hz, 12.0 Hz, 1H), 7.13 (s, 1H), 7.0 (d, J=8.0 Hz, 1H), 6.53 (d, J=8.0 Hz, 1H), 5.0-4.85 (m, 1H), 3.75 (t, J=8.0 Hz, 2H), 3.32 (t, J=8.0 Hz, 2H), 3.0-2.91 (m, 4H), 2.23-2.0 (m, 4H), 1.95-1.75 (m, 5H), 1.57-1.42 (m, 4H), 0.35 (s, 4H).

### Example 5

### N-(1-(4,4-difluorocyclohexyl)-2-oxo-1,2-dihydropyridin-3-yl)-4-((2-hydroxyethyl)sulfonamido)-2-(6-azaspiro[2.5]octan-6-yl)benzamide

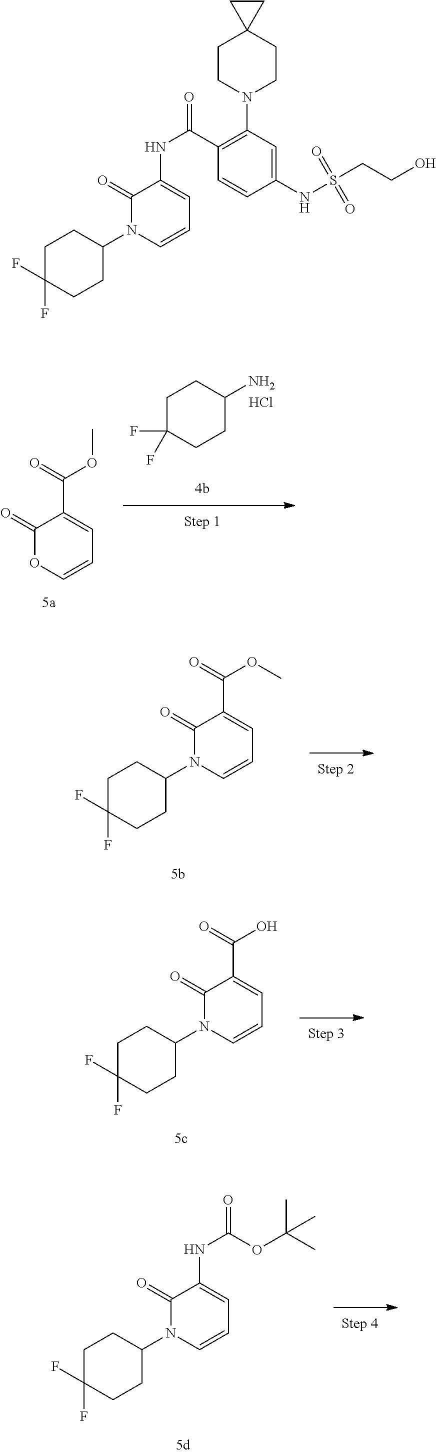

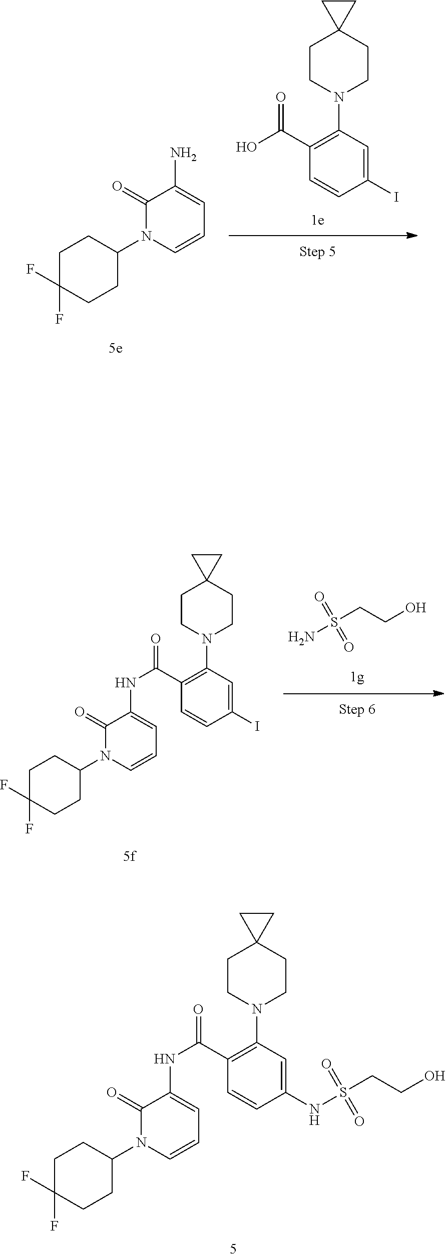 [SMILES: Nc1cccn(C2CCC(F)(F)CC2)c1=O ; O=C(O)c1ccc(I)cc1N1CCC2(CC1)CC2 ; O=C(Nc1cccn(C2CCC(F)(F)CC2)c1=O)c1ccc(I)cc1N1CCC2(CC1)CC2 ; NS(=O)(=O)CCO ; O=C(Nc1cccn(C2CCC(F)(F)CC2)c1=O)c1ccc(NS(=O)(=O)CCO)cc1N1CCC2(CC1)CC2]

### Step 1

### Methyl 1-(4,4-difluorocyclohexyl)-2-oxo-1,2-dihydropyridine-3-carboxylate

4,4-difluorocyclohexne-1-amine hydrochloride 4b (612.44 mg, 3.57 mmol), triethylamine (656.56 mg, 6.49 mmol) and methyl 2-oxo-2H-pyran-3-carboxylate 5a (0.5 g, 3.24 mmol) were added into N,N-dimethylformamide (20 mL) at 0° C. The reaction mixture was stirred at 0-5° C. for 1 hour, then 1-(3-dimethylaminopropyl)-3-ethylcarbodiimide hydrochloride (746.30 mg, 3.89 mmol) and 4-dimethylaminopyridine (118.90 mg, 973.27 μmol) were added, and continuously stirred at room temperature for 16 hours. Ethyl acetate (20 mL) and water (20 mL) were added, the mixture was extracted. The water phase was further extracted with ethyl acetate (20 mL×2), and the organic phases were combined, washed with saturated sodium chloride solution (20 mL), dried over anhydrous sodium sulfate, and concentrated under reduced pressure. The residue was purified by silica gel column chromatography (eluent: system A) to obtain methyl 1-(4,4-difluorocyclohexyl)-2-oxo-1,2-dihydropyridine-3-carboxylate 5b (210 mg) with a yield of 23.86%.

MS m/z (ESI): 272.1 [M+1]

### Step 2

### 1-(4,4-difluorocyclohexyl)-2-oxo-1,2-dihydropyridine-3-carboxylic acid

An aqueous solution (5 mL) of sodium hydroxide (147.46 mg, 3.69 mmol) was added into methyl 1-(4,4-difluorocyclohexyl)-2-oxo-1,2-dihydropyridine-3-carboxylate 5b (200 mg, 737.30 mol) in a mixed solvent of methanol (5 mL) and tetrahydrofuran (5 mL), and stirred at room temperature for 0.5 hours. Hydrochloric acid (1M) was added to adjust a pH to 3-4, ethyl acetate (30 mL) was added and the mixture was extracted. The water phase was further extracted with ethyl acetate (30 mL×2), the organic phases were combined, washed with saturated sodium chloride solution (30 mL), dried over anhydrous sodium sulfate, and concentrated under reduced pressure to obtain 1-(4,4-difluorocyclohexyl)-2-oxo-1,2-dihydropyridine-3-carboxylic acid 5c (180 mg) with a yield of 94.91%.

MS m/z (ESI): 258.1 [M+1]

### Step 3

### Tert-butyl (1-(4,4-difluorocyclohexyl)-2-oxo-1,2-dihydropyridin-3-yl)carbamate

1-(4,4-difluorocyclohexyl)-2-oxo-1,2-dihydropyridin-3-yl) carboxylic acid 5c (450 mg, 1.75 mmol), diphenylphosphoryl azide (529.57 mg, 1.92 mmol) and triethylamine (265.53 mg, 2.62 mmol) were added into tert-butanol (10 mL), and the mixture was heated to 80° C. and stirred for 16 hours. The reaction mixture was concentrated under reduced pressure, and the residue was purified by silica gel column chromatography (eluent: system A) to obtain tert-butyl (1-(4,4-difluorocyclohexyl)-2-oxo-1,2-dihydropyridin-3-yl)carbamate 5d (410 mg) with a yield of 71.38%.

MS m/z (ESI): 273.1 [M-Bu+1]

### Step 4

### 3-Amino-1-(4,4-difluorocyclohexyl)pyridin-2(1H)-one

Tert-butyl (1-(4,4-difluorocyclohexyl)-2-oxo-1,2-dihydropyridin-3-yl)carbamate 5d (110 mg, 335.00 μmol) was added into hydrogen chloride in ethyl acetate (4 M, 10 mL) and stirred at room temperature for 4 hours. The reaction mixture was concentrated under reduced pressure to obtain 3-amino-1-(4,4-difluorocyclohexyl)pyridin-2(1H)-one 5e (88 mg) which was directly used in the next step with a yield of 99%.

MS m/z (ESI): 229.1 [M+1]

### Step 5

### N-(1-(4,4-difluorocyclohexyl)-2-oxo-1,2-dihydropyridin-3-yl)-4-iodo-2-(6-azaspiro[2.5]octan-6-yl)benzamide

3-Amino-1-(4,4-difluorocyclohexyl)pyridin-2(1H)-one 5e (88.93 mg, 335.96 μmol), 4-iodo-2-(6-azaspiro[2.5]octan-6-yl)benzoic acid 1e (100 mg, 279.97 μmol), 1-methylimidazole (69.80 mg, 839.90 μmol) and triethylamine (84.99 mg, 839.90 μmol) were added into acetonitrile (4 mL), and stirred at room temperature for 10 minutes, then N,N,N′,N′-tetramethylchloroformamidine hexafluorophosphate (235.66 mg, 839.90 μmol) was added and the mixture was stirred at room temperature for 4 hours. The reaction mixture was concentrated under reduced pressure, and the residue was purified by silica gel column chromatography (eluent: system A) to obtain N-(1-(4,4-difluorocyclohexyl)-2-oxo-1,2-dihydropyridin-3-yl)-4-iodo-2-(6-azaspiro[2.5]octan-6-yl)benzamide 5f (85 mg), with a yield of 53.51%.

MS m/z (ESI): 568.2 [M+1]

### Step 6

### N-(1-(4,4-difluorocyclohexyl)-2-oxo-1,2-dihydropyridin-3-yl)-4-((2-hydroxyethyl)sulfonamido)-2-(6-azaspiro[2.5]octan-6-yl)benzamide

N-(1-(4,4-difluorocyclohexyl)-2-oxo-1,2-dihydropyridin-3-yl)-4-iodo-2-(6-azaspiro[2.5]octa n-6-yl)benzamide 5f (127.65 mg, 224.97 μmol), 2-hydroxyethane-1-sulfonamide 1g (30.97 mg, 247.47 μmol), 2-(methylamino)acetic acid (30.06 mg, 337.46 μmol), cuprous iodide (12.85 mg, 67.49 μmol) and tripotassium phosphate trihydrate (299.56 mg, 1.12 mmol) were added into N,N-dimethylformamide (3 mL), and the mixture was heated to 110° C. and stirred for 6 hours after argon purging. The reaction mixture was filtered and concentrated, and the residue was purified by preparative liquid chromatography (separation column AKZONOBEL Kromasil; 250×21.2 mm I.D.; 5 μm, 20 mL/min; a mobile phase A: 0.05% TFA+H₂O, and a mobile phase B: CH₃CN) to obtain N-(1-(4,4-difluorocyclohexyl)-2-oxo-1,2-dihydropyridin-3-yl)-4-((2-hydroxyethyl)sulfonamido)-2-(6-azaspiro[2.5]octan-6-yl)benzamide 5 (22 mg) with a yield of 17.09%.

MS m/z (ESI): 565.3 [M+1]

¹H NMR (400 MHz, DMSO-d₆) δ 11.87 (s, 1H), 10.15 (s, 1H), 8.50 (d, J=7.4 Hz, 1H), 7.92 (d, J=8.5 Hz, 1H), 7.47 (d, J=7.1 Hz, 1H), 7.21 (d, J=2.1 Hz, 1H), 7.05 (dd, J=8.5, 2.0 Hz, 1H), 6.34 (t, J=7.2 Hz, 1H), 5.05-4.86 (m, 1H), 3.76 (t, J=6.5 Hz, 2H), 3.35 (t, J=6.5 Hz, 2H), 2.93 (d, J=5.5 Hz, 4H), 2.27-2.06 (m, 4H), 1.94 (dt, J=28.8, 8.8 Hz, 4H), 1.64 (m, 5H), 0.33 (s, 4H).

### Example 6

### N-(3-(4,4-difluoropiperidin-1-yl)-4-methoxyphenyl)-4-((2-hydroxyethyl)sulfonamido)-2-(6-azasp iro[2.5]octan-6-yl)benzamide

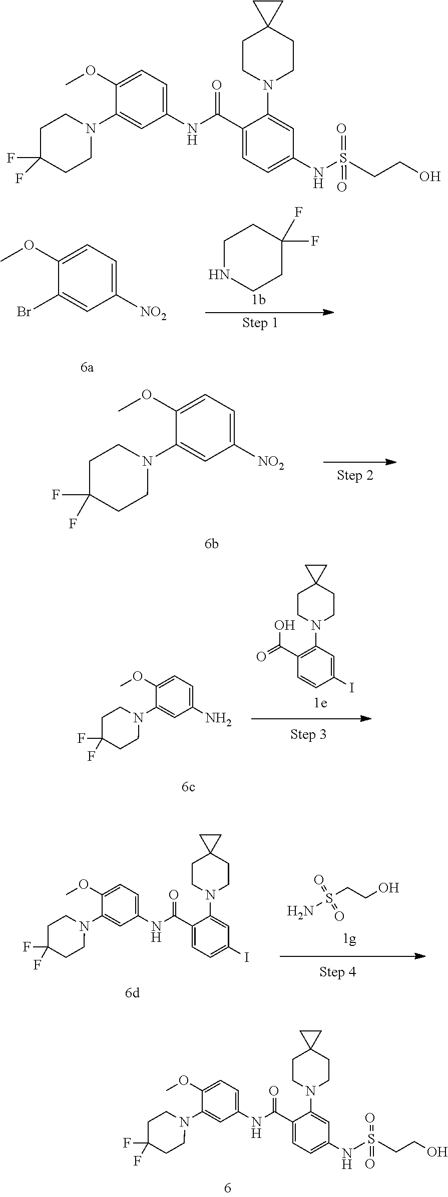

### Step 1

### 4,4-Difluoro-1-(2-methoxy-5-nitrophenyl)piperidine

2-bromo-1-methoxy-4-nitrobenzene 6a (1 g, 4.31 mmol), 4,4-difluorpiperidine 1b (5.17 mmol, 580.04 μL), palladium acetate (48.38 mg, 215.49 μmol), 4,5-bisdiphenylphosphino-9,9-dimethylxanthene (450.74 mg, 861.96 μmol) and cesium carbonate (4.22 g, 12.92 mmol) were dissolved in 1,4-dioxane (10 mL), and the mixture was subjected to argon sparging for 15 minutes, then heated to 100° C. and stirred for 16 hours. Water (20 mL) was added, and the mixture was extracted with ethyl acetate (30 mL×2). The organic phases were combined, washed with saturated sodium chloride (20 mL), dried over anhydrous sodium sulfate, filtered and concentrated. The residue was purified by silica gel chromatography (eluent: system A) to obtain 4,4-difluoro-1-(2-methoxy-5-nitrophenyl)piperidine 6b (1.1 g) with a yield of 93.75%.

MS m/z (ESI): 273.3 [M+1]

### Step 2

### 3-(4,4-Difluoropiperidin-1-yl)-4-methoxyaniline

4,4-Difluoro-1-(2-methoxy-5-nitrophenyl)piperidine 6b (1.1 g, 4.04 mmol) and10% palladium carbon (129.00 mg, 1.21 mmol) was added into methanol (2 mL), the mixture was subjected to hydrogen purging for three times and then reacted at room temperature for 3 hours. The reaction mixture was filtered and concentrated under a reduced pressure. The residue was purified by silica gel chromatography (eluent: system A) to obtain 3-(4,4-difluoropiperidin-1-yl)-4-methoxyaniline 6c (600 mg) with a yield of 61.3%.

MS m/z (ESI): 243.1 [M+1]

### Step 3

### N-(3-(4,4-difluoropiperidin-1-yl)-4-methoxyphenyl)-4-iodo-2-(6-azaspiro[2.5]octan-6-yl)benzam ide

4-iodo-2-(6-azaspiro[2.5]octan-6-yl)benzoic acid 1e (405.45 mg, 1.14 mmol, self-prepared according to the method in published patent WO2020132648), N-methylimidazole (254.16 mg, 3.10 mmol) and 3-(4,4-difluoropiperidin-1-yl)-4-methoxyphenyl 6c (250 mg, 1.03 mmol) were added into acetonitrile (5 mL), the mixture was stirred for 5 minutes, and then N,N,N′,N′-tetramethylchloroformamidine hexafluorophosphate (866.82 mg, 3.10 mmol) was added and reacted for 3 hours. Water (20 mL) was added and the mixture was extracted with ethyl acetate (30 mL×2). The organic phases were combined, washed with saturated sodium chloride (10 mL), dried over anhydrous sodium sulfate, filtered and concentrated. The residue was purified by silica column chromatography (eluent: system A) to obtain N-(3-(4,4-difluoropiperidin-1-yl)-4-methoxyphenyl)-4-iodo-2-(6-azaspiro[2.5]octan-6-yl)benzam ide 6d (150 mg) with a yield of 25%.

MS m/z (ESI): 582.4 [M+1]

### Step 4

### N-(3-(4,4-difluoropiperidin-1-yl)-4-methoxyphenyl)-4-((2-hydroxyethyl)sulfonamido)-2-(6-azasp iro[2.5]octan-6-yl)benzamide

2-hydroxyethne-1-sulfonamide 1g (32.29 mg, 257.98 μmol) and N-(3-(4,4-difluoropiperidin-1-yl)-4-methoxyphenyl)-4-iodo-2-(6-azaspiro[2.5]octan-6-yl)benzam ide 6d (150 mg, 257.98 μmol) were added into N,N-dimethylformamide(3 mL), then 2-(methylamino)acetic acid (22.98 mg, 257.98 μmol), tripotassium phosphate trihydrate (164.29 mg, 773.95 μmol) and cuprous iodide (28.64 mg, 128.99 μmol) were added, and the mixture was heated to 110° C. to react for 3 hours under argon protection. After cooling, ethyl acetate (20 mL) and water (10 mL) were added to the reaction mixture and extracted. The organic phases were concentrated. The residue was purified by preparative liquid chromatography (separation column AKZONOBEL Kromasil; 250×21.2 mm I.D.; 5 μm, 20 mL/min; a mobile phase A: 0.05% TFA+H₂O, and a mobile phase B: CH₃CN) to obtain N-(3-(4,4-difluoropiperidin-1-yl)-4-methoxyphenyl)-4-((2-hydroxyethyl)sulfonamido)-2-(6-azasp iro[2.5]octan-6-yl)benzamide 6 (50 mg) with a yield of 31.82%.

MS m/z (ESI): 579.3 [M+1]

¹H NMR (400 MHz, DMSO-d₆) δ 11.61 (s, 1H), 10.07 (s, 1H), 7.84 (d, J=8.5 Hz, 1H), 7.43 (d, J=2.4 Hz, 1H), 7.38 (dd, J=8.8, 2.4 Hz, 1H), 7.16 (d, J=2.1 Hz, 1H), 7.03 (dd, J=8.4, 2.0 Hz, 1H), 6.97 (d, J=8.7 Hz, 1H), 4.94 (t, J=5.6 Hz, 1H), 3.79 (s, 3H), 3.78-3.72 (m, 2H), 3.33 (d, J=6.9 Hz, 2H), 3.11 (t, J=5.7 Hz, 4H), 2.96 (t, J=5.3 Hz, 4H), 2.13 (d, J=15.5 Hz, 4H), 1.55 (s, 4H), 0.35 (s, 4H).

### Example 7

### N-(4-cyano-3-(4,4-difluoropiperidin-1-yl)phenyl)-4-((2-hydroxyethyl)sulfonamido)-2-(6-azaspiro [2.5]octan-6-yl)benzamide

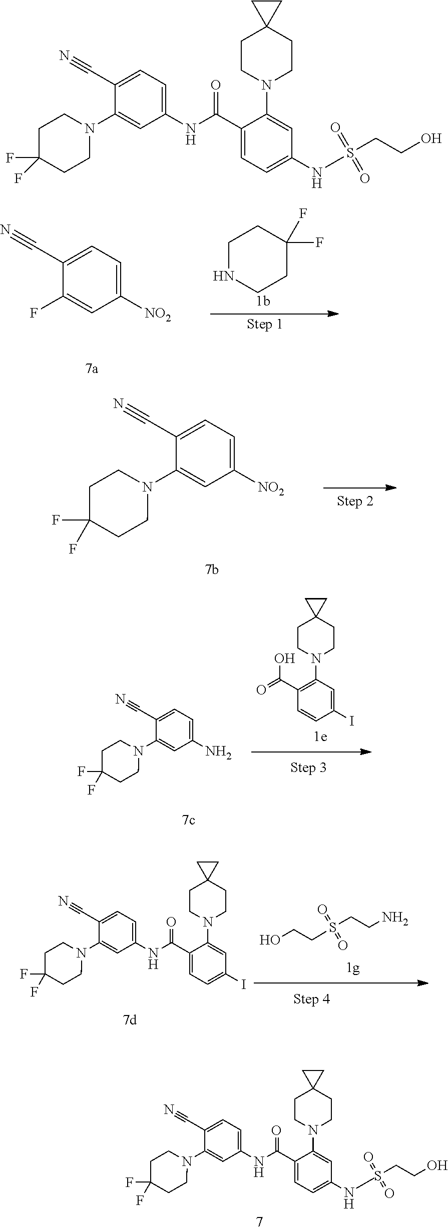

### Step 1

### 2-(4,4-Difluoropiperidin-1-yl)-4-nitrobenzonitrile

2-fluoro-4-nitrobenzonitrile 7a (1 g, 6.02 mmol), 4,4-difluorpiperidine 1b (802.13 mg, 6.62 mmol) and potassium carbonate (2.50 g, 18.06 mmol) were added into N,N-dimethylformamide (5 mL), the mixture was heated to 80° C. and stirred for 4 hours. Water (20 mL) was added and the mixture was extracted with ethyl acetate (30 mL×2). The organic phases were combined, washed with saturated sodium chloride (20 mL), dried over anhydrous sodium sulfate, filtered, and concentrated. The residue was dissolved with ethyl acetate, and petroleum ether was added to precipitate 2-(4,4-difluoropiperidin-1-yl)-4-nitrobenzonitrile 7b (1.0 g) as a solid with a yield of 62.16%.

MS m/z (ESI): 268.1 [M+1]

### Step 2

### 4-Amino-2-(4,4-difluoropiperidin-1-yl)benzonitrile

2-(4,4-Difluoropiperidin-1-yl)-4-nitrobenzonitrile 7b (1.0 g, 3.74 mmol) and 10% palladium carbon (119.47 mg, 1.12 mmol) was added in methanol (10 mL), and the mixture was subjected to hydrogen purging for three times and then stirred at room temperature for 6 hours. The reaction mixture was filtered and concentrated under reduced pressure. The residue was purified by silica gel chromatography (eluent: system A) to obtain 4-amino-2-(4,4-difluoropiperidin-1-yl)benzonitrile 7c (850 mg) with a yield of 95.74%.

MS m/z (ESI): 238.1 [M+1]

### Step 3

### N-(4-cyano-3-(4,4-difluoropiperidin-1-yl)phenyl)-4-iodo-2-(6-azaspiro[2.5]octan-6-yl)benzamide

4-iodo-2-(6-azaspiro[2.5]octane-6-yl) benzoic acid 1e (662.44 mg, 1.85 mmol) and 4-amino-2-(4,4-difluoropiperidine-1-yl)benzonitrile 7c (400 mg, 1.69 mmol) were added into 1,4-dioxane (1 mL), then N,N,N′,N′-tetramethylchloroformamidine hexafluorophosphate (1.42 g, 5.06 mmol) and N-methylimidazole (415.26 mg, 5.06 mmol) were added, and then the mixture was heated to 100° C. to react for 3 hours. Water (20 mL) was added and the mixture was extracted with ethyl acetate (30 mL×2). The organic phases were combined, washed with saturated sodium chloride (10 mL), dried over anhydrous sodium sulfate, filtered and concentrated. The residue was purified by silica column chromatography (eluent: system A) to obtain N-(4-cyano-3-(4,4-difluoropiperidin-1-yl)phenyl)-4-iodo-2-(6-azaspiro[2.5]octan-6-yl)benzamide 7d (700 mg) with a yield of 72.03%.

MS m/z (ESI): 557.2 [M+1]

1H NMR (400 MHz, Chloroform-d) δ 12.83 (s, 1H), 7.97 (d, J=8.5 Hz, 1H), 7.90 (d, J=1.9 Hz, 1H), 7.72-7.66 (m, 2H), 7.56 (d, J=8.4 Hz, 1H), 7.16 (d, J=8.4 Hz, 1H), 3.38 (t, J=5.6 Hz, 4H), 3.09 (t, J=5.4 Hz, 4H), 2.22 (tt, J=13.2, 5.5 Hz, 4H), 1.66 (s, 4H), 0.46 (s, 4H).

### Step 4

### N-(4-cyano-3-(4,4-difluoropiperidin-1-yl)phenyl)-4-((2-hydroxyethyl)sulfonamido)-2-(6-azaspiro [2.5]octan-6-yl)benzamide

2-hydroxyethane-1-sulfonamide 1g (21.71 mg, 173.49 μmol) and N-(4-cyano-3-(4,4-difluoropiperidin-1-yl)phenyl)-4-iodo-2-(6-azaspiro[2.5]octan-6-yl)benzamide 7d (100 mg, 173.49 μmol) were dissolved in N,N-dimethylformamide(5 mL), then 2-(methylamino)acetic acid (15.46 mg, 173.49 μmol), tripotassium phosphate trihydrate (110.48 mg, 520.46 mol) and cuprous iodide (19.26 mg, 86.74 μmol) were added, and the mixture was subjected to argon sparging for 15 minutes, heated to 100° C. and stirred for 10 hours. After cooling, ethyl acetate (20 mL) and water (10 mL) were added, and the mixture was extracted. The organic phases were concentrated. The residue was purified by preparative liquid chromatography (separation column AKZONOBEL Kromasil; 250×21.2 mm I.D.; 5 μm, 20 mL/min; a mobile phase A: 0.05% NH₄HCO₃+H₂O, and a mobile phase B: CH₃CN) to obtain N-(4-cyano-3-(4,4-difluoropiperidin-1-yl)phenyl)-4-((2-hydroxyethyl)sulfonamido)-2-(6-azaspiro [2.5]octan-6-yl)benzamide 7 (31 mg) with a yield of 30.21%.

MS m/z (ESI): 574.2 [M+1]

¹H NMR (400 MHz, DMSO-d₆) δ 11.89 (s, 1H), 7.79 (d, J=8.5 Hz, 1H), 7.75-7.69 (m, 2H), 7.46 (dd, J=8.4, 1.8 Hz, 1H), 7.15 (d, J=2.1 Hz, 1H), 7.02 (dd, J=8.5, 2.1 Hz, 1H), 3.76 (t, J=6.5 Hz, 2H), 3.42-3.39 (m, 2H), 3.32-3.27 (m, 4H), 2.96 (t, J=5.1 Hz, 4H), 2.16 (dq, J=13.5, 6.9, 5.4 Hz, 4H), 1.51 (s, 4H), 0.34 (s, 4H).

Examples 8-10 were synthesized according to the synthesis methods of Examples 6-7 of the present invention. Spectrum parameters of Examples 8-10 can be seen from the following table:

| | MS m/z | |
|---|---|---|
| Example No. and Structure | (ESI) | ¹H NMR |
| 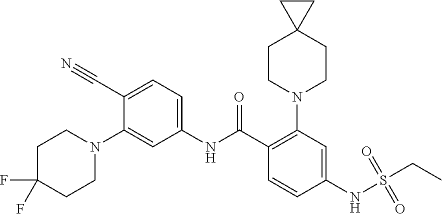 [SMILES: CCS(=O)(=O)Nc1ccc(C(=O)Nc2ccc(C#N)c(N3CCC(F)(F)CC3)c2)c(N2CCC3(CC2)CC3)c1] | 558.6 [M + H] | 1H NMR (400 MHz, ) δ 11.83 (s, 1H), 10.19 (s, 1H), 7.82-7.68 (m, 3H), 7.50-7.42 (m, 1H), 7.16 (d, J = 2.0 Hz, 1H), 7.03 (dd, J = 8.5, 2.1 Hz, 1H), 3.28 (d, J = 6.6 Hz, 4H), 3.20 (q, J = 7.3 Hz, 2H), 2.96 (t, J = 5.2 Hz, 4H), 2.22-2.12 (m, 4H), 1.51 (s, 4H), 1.21 (t, J = 7.3 Hz, 3H), 0.34 (s, 4H). |
| 8 | | |
| 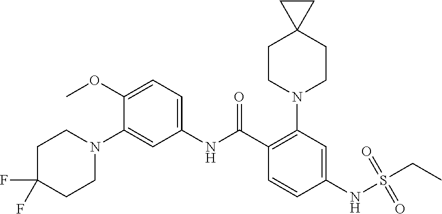 [SMILES: CCS(=O)(=O)Nc1ccc(C(=O)Nc2ccc(OC)c(N3CCC(F)(F)CC3)c2)c(N2CCC3(CC2)CC3)c1] | 562.9 [M + 1] | 1H NMR (400 MHz, DMSO-d6) δ 11.60 (s, 1H), 10.13 (s, 1H), 7.83 (d, J = 8.5 Hz, 1H), 7.46-7.34 (m, 2H), 7.17 (d, J = 2.1 Hz, 1H), 7.04 (dd, J = 8.5, 2.1 Hz, 1H), 6.97 (d, J = 8.8 Hz, 1H), 3.79 (s, 3H), 3.19 (q, J = 7.3 Hz, 2H), 3.10 (t, J = 5.6 Hz, 4H), 2.96 (t, J = 5.2 Hz, 4H), 2.12 (td, J = 14.1, 13.5, 6.4 Hz, 4H), 1.55 (t, J = 4.9 Hz, 4H), 1.20 (t, J = 7.3 Hz, 3H), 0.35 (s, 4H). |
| 9 | | |
| 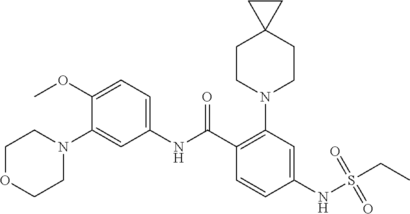 [SMILES: CCS(=O)(=O)Nc1ccc(C(=O)Nc2ccc(OC)c(N3CCOCC3)c2)c(N2CCC3(CC2)CC3)c1] | 529.1 [M + 1] | 1H NMR (400 MHz, ) δ 11.68 (s, 1H), 10.22 (s, 1H), 7.84 (d, J = 8.5 Hz, 1H), 7.42-7.33 (m, 2H), 7.20 (d, J = 2.1 Hz, 1H), 7.06 (dd, J = 8.5, 2.1 Hz, 1H), 6.96 (d, J = 8.6 Hz, 1H), 3.78 (s, 3H), 3.74 (t, J = 4.5 Hz, 4H), 3.18 (q, J = 7.3 Hz, 2H), 2.97 (m, 8H), 1.56 (s, 4H), 1.21 (t, J = 7.3 Hz, 3H), 0.36 (s, 4H). |
| 10 | | |

### Example 11

### N-(3-((3,3-difluoroazetidin-1-yl)methyl)-4-methoxyphenyl)-4-(ethylsulfonamido)-2-(6-azaspiro[2.5]octan-6-yl)benzamide

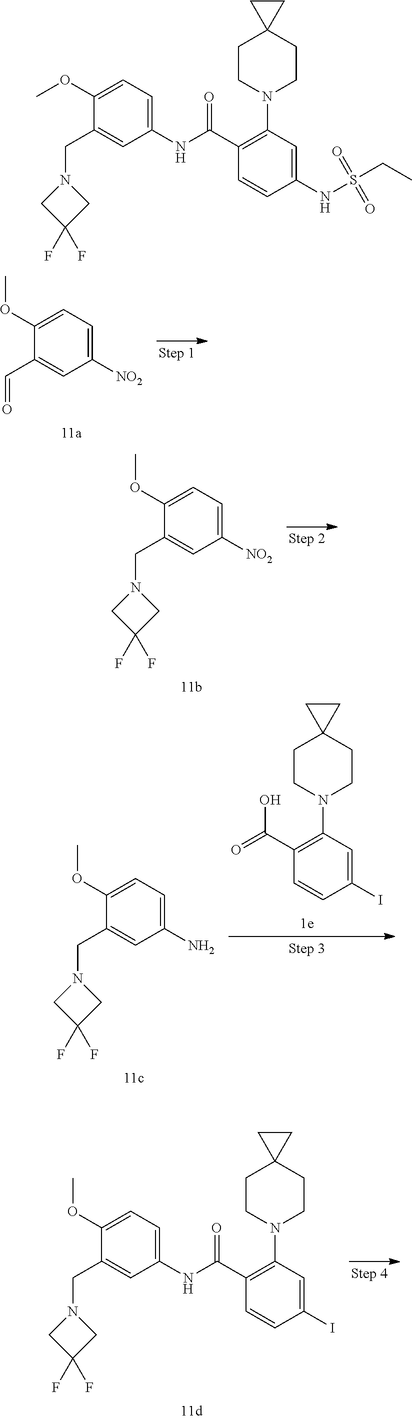 [SMILES: CCS(=O)(=O)Nc1ccc(C(=O)Nc2ccc(OC)c(CN3CC(F)(F)C3)c2)c(N2CCC3(CC2)CC3)c1 ; COc1ccc([N+](=O)[O-])cc1C=O ; COc1ccc([N+](=O)[O-])cc1CN1CC(F)(F)C1 ; COc1ccc(N)cc1CN1CC(F)(F)C1 ; O=C(O)c1ccc(I)cc1N1CCC2(CC1)CC2 ; COc1ccc(NC(=O)c2ccc(I)cc2N2CCC3(CC2)CC3)cc1CN1CC(F)(F)C1]

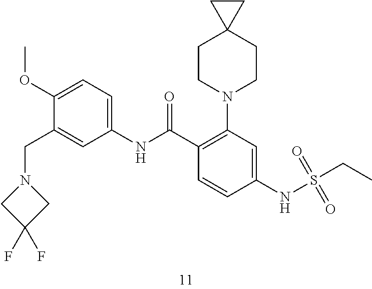 [SMILES: CCS(=O)(=O)Nc1ccc(C(=O)Nc2ccc(OC)c(CN3CC(F)(F)C3)c2)c(N2CCC3(CC2)CC3)c1]

### Step 1

### 3,3-Difluoro-1-(2-methoxy-5-nitrobenzyl)azetidine

Acetic acid (828.76 mg, 13.80 mmol, 789.29 μL) was added into 2-methoxy-5-nitrobenzaldehyde 11a (500 mg, 2.76 mmol, commercially available) in methanol (2 mL) solution, and stirred at room temperature for five minutes, and then 3,3-difluorazetidine (393.30 mg, 3.04 mmol) was added. After stirring for half an hour, sodium cyanoborohydride (867.26 mg, 13.80 mmol) was added, and the mixture was stirred at room temperature for half an hour. The reaction mixture was poured into water (10 mL) and extracted with ethyl acetate (30 mL×3). The organic phases were combined, washed with saturated sodium chloride solution (50 mL), dried over anhydrous sodium sulfate and concentrated under reduced pressure. The residue was purified by silica gel column chromatography (eluent: system A) to obtain 3,3-difluoro-1-(2-methoxy-5-nitrobenzyl)azetidine 11b (200 mg) with a yield of 28.06%.

MS m/z (ESI): 259.1 [M+1]

### Step 2

### 3-((3,3-Difluoroazetidin-1-yl)methyl)-4-methoxyaniline

At room temperature, zinc powder (253.23 mg, 3.87 mmol) and ammonium chloride (207.15 mg, 3.87 mmol) were added into 3,3-difluoro-1-(2-methoxy-5-nitrobenzyl)azetidine 11b (200 mg, 774.53 μmol) in a mixed solvent of ethanol (2 mL) and water (0.5 mL), and the mixture was stirred at 70° C. for 3 hours. The reaction mixture was poured into water (10 mL) and extracted with ethyl acetate (30 mL×3). The organic phases were combined, washed with saturated sodium chloride solution (50 mL), dried over anhydrous sodium sulfate and concentrated under reduced pressure to obtain 3-((3,3-difluoroazetidin-1-yl)methyl)-4-methoxyaniline 11c (170 mg) with a yield of 96.2%.

MS m/z (ESI): 229.2 [M+1]

### Step 3

### N-(3-((3,3-difluoroazetidin-1-yl)methyl)-4-methoxyphenyl)-4-iodo-2-(6-azaspiro[2.5]octan-6-yl) benzamide

At room temperature, N,N,N′,N′-tetramethylchloroformamidine hexafluorophosphate (552.06 mg, 1.97 mmol) was added into a solution of 4-iodo-2-(6-azaspiro[2.5]octan-6-yl)benzoic acid 1e (234.75 mg, 657.21 μmol) and N-methylimidazole (161.87 mg, 1.97 mmol) in acetonitrile (2 mL), and the mixture was stirred at room temperature for five minutes, then 3-((3,3-difluorazetidin-1-yl)methyl)-4-methoxyaniline 11c (150 mg, 657.21 μmol) was added and the mixture was stirred at room temperature for three hours. The reaction mixture was poured into water (10 mL), and extracted with ethyl acetate (30 mL×3)ₒ The organic phases were combined, washed with saturated sodium chloride solution (50 mL), dried over anhydrous sodium sulfate and concentrated under reduced pressure. The residue was purified by silica gel column chromatography (eluent: system A) to obtain N-(3-((3,3-difluoroazetidin-1-yl)methyl)-4-methoxyphenyl)-4-iodo-2-(6-azaspiro[2.5]octan-6-yl) benzamide 11d (200 mg) with a yield of 53.6%.

MS m/z (ESI): 569.1 [M+1]

### Step 4

### N-(3-((3,3-difluoroazetidin-1-yl)methyl)-4-methoxyphenyl)-4-(ethylsulfonamido)-2-(6-azaspiro[2.5]octan-6-yl)benzamide

At room temperature, ethyl sulfonamide (42.32 mg, 387.73 μmol) and N-(3-((3,3-difluoroazetidin-1-yl)methyl)-4-methoxyphenyl)-4-iodo-2-(6-azaspiro[2.5]octan-6-yl) benzamide 11d (200 mg, 352.48 μmol) were added into potassium phosphate (224.46 mg, 1.06 mmol), cuprous iodide (39.14 mg, 176.24 μmol) and sarcosine (31.40 mg, 352.48 μmol) in N,N-dimethylformamide (1 mL), and the mixture was subjected to argon purging for three times and stirred at 110° C. for 3 hours. The reaction mixture was poured into water (10 mL) and extracted with ethyl acetate (30 mL×3). The organic phases were combined, washed with saturated sodium chloride solution (50 mL), dried over anhydrous sodium sulfate, and concentrated under reduced pressure. The residue was purified by preparative liquid chromatography (separation column AKZONOBEL Kromasil; 250×21.2 mm I.D.; 5 μm, 20 mL/min; a mobile phase A: 0.05% NHHCO₃+H₂O, a mobile phase B: CH₃CN) to obtain N-(3-((3,3-difluoroazetidin-1-yl)methyl)-4-methoxyphenyl)-4-(ethylsulfonamido)-2-(6-azaspiro[ 2.5]octan-6-yl)benzamide 11 (36.54 mg), with a yield of 18.44%.

MS m/z (ESI): 549.3 [M+1]

1H NMR (400 MHz) δ 11.53 (s, 1H), 10.13 (s, 1H), 7.85-7.78 (m, 2H), 7.53 (d, J=2.6 Hz, 1H), 7.17 (d, J=2.1 Hz, 1H), 7.07-6.95 (m, 2H), 3.78 (s, 3H), 3.69 (d, J=24.8, 4H), 3.67 (d, J=12.3 Hz, 2H), 3.19 (q, J=7.3 Hz, 2H), 2.97 (t, J=5.2 Hz, 4H), 1.53 (s, 4H), 1.21 (t, J=7.3 Hz, 3H), 0.35 (s, 4H).

Example 12 was synthesized according to the synthesis method of Example 11 of the present invention. Spectrum parameters of Example 12 can be seen from the following table:

| | MS m/z | |
|---|---|---|
| Example No. and Structure | (ESI) | 1H NMR |
| 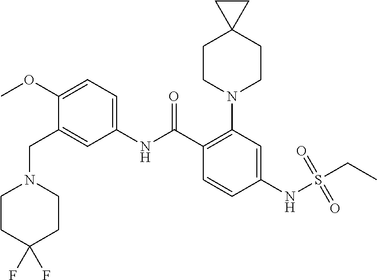 [SMILES: CCS(=O)(=O)Nc1ccc(C(=O)Nc2ccc(OC)c(CN3CCC(F)(F)CC3)c2)c(N2CCC3(CC2)CC3)c1] | 577.3 [M + 1] | 1H NMR (400 MHz, DMSO-d6) δ 11.46 (s, 1H), 10.14 (s, 1H), 8.17 (s, 1H), 7.80 (d, J = 8.5 Hz, 1H), 7.58 (d, J = 8.8 Hz, 1H), 7.22 (d, J = 9.0 Hz, 1H), 7.17 (d, J = 2.1 Hz, 1H), 7.04 (dd, J = 8.6, 2.1 Hz, 1H), 4.41 (s, 2H), 3.87 (s, 3H), 3.53-3.50 (m, 2H), 3.27-3.25 (m, 2H), 3.18 (q, J = 7.3 Hz, 2H), 2.98 (t, J = 5.3 Hz, 4H), 2.35-2.25 (m, 4H), 1.51 (s, 4H), 1.20 (d, J = 7.3 Hz, 3H), 0.35 (s, 4H). |
| 12 | | |

### Example 13

### N-(3-(4,4-difluoropiperidin-1-yl)-4-(trifluoromethoxy)phenyl)-4-(ethylsulfonamido)-2-(6-azaspir o[2.5]octan-6-yl)benzamide

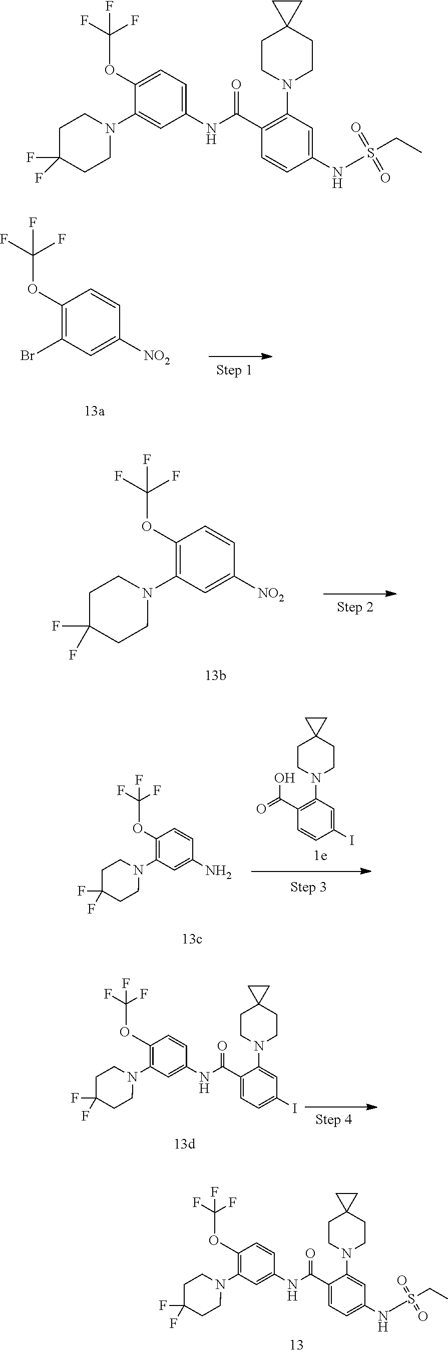

### Step 1

### 4,4-difluoro-1-(5-nitro-2-(trifluoromethoxy)phenyl)piperidine

At room temperature, 2-bromo-4-nitro-1-(trifluoromethoxy)benzene 13a (350 mg, 1.22 mmol, commercially available) and 4,4-difluorpiperidine (296.46 mg, 2.45 mmol) were dissolved in toluene (2 mL), then tris(dibenzylideneacetone)dipalladium (112.06 mg, 122.38 μmol), 2-dicyclohexylphosphine-2′,4′,6′-triisopropylbiphenyl (116.68 mg, 244.75 μmol) and cesium carbonate (1.20 g, 3.67 mmol) were added, and the mixture was subjected to argon purging and stirred at 100° C. for 20 hours. Mass spectrometry showed that the reaction was completed. Water (20 mL) was added, and the mixture was extracted with ethyl acetate (30 mL×3). The organic phases were combined, washed with saturated sodium chloride solution, dried over anhydrous sodium sulfate, filtered and concentrated under reduced pressure. The residue was purified by silica gel column chromatography (eluent: system A) to obtain 4,4-difluoro-1-(5-nitro-2-(trifluoromethoxy)phenyl)piperidine 13b (0.38 g) with a yield of 95.19%.

MS m/z (ESI): 326.9 [M+1]

### Step 2

### 3-(4,4-Difluoropiperidin-1-yl)-4-(trifluoromethoxy)aniline

At room temperature, 4,4-difluoro-1-(5-nitro-2-(trifluoromethoxy)phenyl)piperidine 13b (380 mg, 1.16 mmol) and palladium carbon (123.96 mg, 1.16 mmol, 10%) was added in methanol (20 mL), then the mixture was subjected to hydrogen purging for three times and stirred at 60° C. for 6 hours. Mass spectrometry showed that the reaction was completed. The reaction mixture was filtered under reduced pressure, and the filtrate was concentrated to obtain 3-(4,4-difluoropiperidin-1-yl)-4-(trifluoromethoxy)aniline 13c (340 mg) with a yield of 98.53%.

MS m/z (ESI): 296.9 [M+1]

### Step 3

### N-(3-(4,4-difluoropiperidin-1-yl)-4-(trifluoromethoxy)phenyl)-4-iodo-2-(6-azaspiro[2.5]octan-6-yl)benzamide

At room temperature, 4-iodo-2-(6-azaspiro[2.5]octan-6-yl) benzoic acid 1e (100 mg, 279.97 μmol) was dissolved in acetonitrile (10 mL), then 1-methylimidazole (45.97 mg, 559.93 mol) and N,N,N′,N′-tetramethylchloroformamidine hexafluorophosphate (157.11 mg, 559.93 μmol) were added, and the mixture was stirred at room temperature for 1 hour, then (3-(4,4-difluoropiperidin-1-yl)-4-(trifluoromethoxy)aniline 13c (107.82 mg, 363.96 mol) was added, the mixture was heater to 60° C. and stirred for 1 hour. Mass spectrometry showed that the reaction was completed. After concentration under reduced pressure, the residue was purified by silica gel column chromatography (eluent: system A) to obtain N-(3-(4,4-difluoropiperidin-1-yl)-4-(trifluoromethoxy)phenyl)-4-iodo-2-(6-azaspiro[2.5]octan-6-yl)benzamide 13d (140 mg) with a yield of 78.70%.

MS m/z (ESI): 636.2 [M+1]

### Step 4

### N-(3-(4,4-difluoropiperidin-1-yl)-4-(trifluoromethoxy)phenyl)-4-(ethylsulfonamido)-2-(6-azaspir o[2.5]octan-6-yl)benzamide

At room temperature, N-(3-(4,4-difluoropiperidin-1-yl)-4-(trifluoromethoxy)phenyl)-4-iodo-2-(6-azaspiro[2.5]octan-6-yl)benzamide 13d (140 mg, 220.33 μmol) and ethyl sulfonamide (48.10 mg, 440.66 μmol) were dissolved in N,N-dimethylformamide (10 mL), then cuprous iodide (24.46 mg, 110.17 μmol), 2-(methylamino)acetic acid (19.63 mg, 220.33 mol) and potassium phosphate (293.38 mg, 1.10 mmol) were added, and the mixture was subjected to argon purging, heated to 110° C., and stirred for 6 hours. Mass spectrometry showed that the reaction was completed. Water (20 mL) was added, the mixture was extracted with ethyl acetate (30 mL×3). The organic phases were combined and washed with saturated sodium chloride solution, dried over anhydrous sodium sulfate, filtered, and concentrated under reduced pressure. The residue was purified by preparative liquid chromatography (separation column AKZONOBEL Kromasil; 250×21.2 mm I.D.; 5 μm, 20 mL/min; a mobile phase A: 0.05% NH₄CO₃+H₂O, a mobile phase B: CH₃CN) to obtain N-(3-(4,4-difluoropiperidin-1-yl)-4-(trifluoromethoxy)phenyl)-4-(ethylsulfonamido)-2-(6-azaspir o[2.5]octan-6-yl)benzamide 13 (60.9 mg) with a yield of 44.64%.

MS m/z (ESI): 617.3 [M+1]

1H NMR (400 MHz, DMSO-d6) δ 11.74 (s, 1H), 10.11 (s, 1H), 7.81 (d, J=8.4 Hz, 1H), 7.67 (d, J=2.5 Hz, 1H), 7.45 (dd, J=8.8, 2.4 Hz, 1H), 7.32 (dd, J=8.8, 1.5 Hz, 1H), 7.17 (d, J=2.1 Hz, 1H), 7.04 (dd, J=8.5, 2.1 Hz, 1H), 3.24-3.17 (m, 2H), 3.17-3.10 (m, 4H), 2.97 (t, J=5.2 Hz, 4H), 2.18-2.05 (m, 4H), 1.60-1.45 (m, 4H), 1.21 (t, J=7.3 Hz, 3H), 0.35 (s, 4H).

### Example 14

### N-(4′,6-dimethoxy-[1,1′-biphenyl]-3-yl)-4-(ethylsulfonamido)-2-(6-azaspiro[2.5]octan-6-yl)benza mide

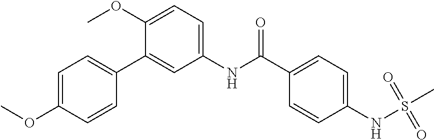

### Step 1

### 4-(ethylsulfonamido)-2-(6-azaspiro[2.5]octan-6-yl)benzoic acid

4-iodo-2-(6-azaspiro[2.5]octan-6-yl)benzoic acid 1e (1.0 g, 2.80 mmol) and ethyl sulfonamide (458.36 mg, 4.20 mmol) were dissolved in N,N-dimethylformamide (10 mL), then cuprous iodide (266.60 mg, 1.40 mmol), sarcosine (249.43 mg, 2.80 mmol) and potassium phosphate (3.73 g, 14.00 mmol) were added, the mixture was subjected to argon purging, heated to 110° C. and stirred for 6 hours. After the reaction was completed, the reaction system was cooled to room temperature, the reaction mixture was poured into ice water (200 mL), and adjusted pH to 6 with diluted hydrochloric acid (2 M), then dichloromethane (200 mL×3) was added for extraction. The organic phases were combined and washed with saturated sodium chloride solution (50 mL), dried over anhydrous sodium sulfate, filtered, concentrated under reduced pressure. The residue was purified by silica gel column chromatography (eluent: system A) to obtain 4-(ethylsulfonamido)-2-(6-azaspiro[2.5]octan-6-yl)benzoic acid 14a (0.75 g) with a yield of 79.16%.

MS m/z (ESI): 339.4 [M+1]

### Step 2

### 4′,6-Dimethoxy-[1,1′-biphenyl]-3-amine

At room temperature, 3-bromo-4-methoxyaniline 14b (300 mg, 1.48 mmol, commercially available), (4-methoxyphenyl)boronic acid 14c (248.18 mg, 1.63 mmol, commercially available), [1,1′-bis(diphenylphosphino)ferrocene]palladium dichloride (107.75 mg, 148.48 mol) and cesium carbonate (967.55 mg, 2.97 mmol) were added into a mixed solution of 1,4-dioxane(2 mL) and water (1 mL), and the mixture was subjected to argon purging for three times and stirred at 100° C. for 3 hours. After the reaction was completed, the reaction mixture was poured into water (30 mL) and extracted with ethyl acetate (30 mL×3). The organic phases were combined and washed with saturated sodium chloride solution (50 mL), dried over anhydrous sodium sulfate, and concentrated under reduced pressure. The residue was purified by silica gel column chromatography (eluent: A system) to obtain 4′,6-dimethoxy-[1,1′-biphenyl]-3-amine 14d (300 mg) with a yield of 88.13%.

MS m/z (ESI): 339.4 [M+1]

### Step 3

### N-(4′,6-dimethoxy-[1,1′-biphenyl]-3-yl)-4-(ethylsulfonamido)-2-(6-azaspiro[2.5]octan-6-yl)benza mide

At room temperature, 4-(ethylsulfonamido)-2-(6-azaspiro[2.5]octan-6-yl)benzoic acid 14a (50 mg, 147.74 μmol) and triethylamine (44.85 mg, 443.23 mol) were added into 1H-benzotriazole-1-yloxytripyrrolidine hexafluorophosphate (76.89 mg, 147.74 μmol) in N,N-dimethylformamide (1 mL), and the mixture was stirred at room temperature for 30 minutes, then 4′,6-dimethoxy-[1,1′-biphenyl]-3-amine 14d (44.04 mg, 192.07 μmol) was added and the mixture was stirred at room temperature for 3 hours. After the reaction was completed, the reaction mixture was poured into water (50 mL), extracted with ethyl acetate (30 mL×3). The organic phases were combined and washed with saturated sodium chloride solution (50 mL), dried over anhydrous sodium sulfate, and concentrated under reduced pressure. The residue was separated by preparative liquid chromatography (separation column AKZONOBEL Kromasil; 250×21.2 mm I.D.; 5 μm, 20 mL/min; a mobile phase A: 0.05% NH₄HCO₃+H₂O, a mobile phase B: CH₃CN) to obtain N-(4′,6-dimethoxy-[1,1′-biphenyl]-3-yl)-4-(ethylsulfonamido)-2-(6-azaspiro[2.5]octan-6-yl)benza mide 14 (15 mg) with a yield of 13.54%.

MS m/z (ESI): 550.2 [M+1]

1H NMR (400 MHz, DMSO-d6) δ 11.49 (s, 1H), 10.13 (s, 1H), 7.81 (d, J=8.5 Hz, 1H), 7.74 (d, J=2.7 Hz, 1H), 7.65 (dd, J=8.8, 2.7 Hz, 1H), 7.45-7.41 (m, 2H), 7.17-7.10 (m, 2H), 7.05-6.98 (m, 3H), 3.80 (s, 3H), 3.75 (s, 3H), 3.19 (q, J=7.3 Hz, 2H), 2.98 (t, J=5.3 Hz, 4H), 1.54 (t, J=5.0 Hz, 4H), 1.21 (t, J=7.3 Hz, 3H), 0.36 (s, 4H).

Examples 15-16 were synthesized according to the synthesis method of Example 14 of the present invention. Spectrum parameters of Examples 15-16 can be seen from the following table:

| | MS m/z | |
|---|---|---|
| Example No. and Structure | (ESI) | 1H NMR |
| 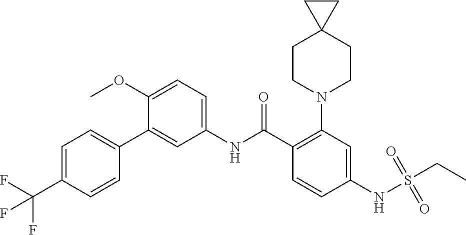 [SMILES: CCS(=O)(=O)Nc1ccc(C(=O)Nc2ccc(OC)c(-c3ccc(C(F)(F)F)cc3)c2)c(N2CCC3(CC2)CC3)c1] | 587.8 [M + 1] | 1H NMR (400 MHz, DMSO-d6) δ 11.47 (s, 1H), 10.12 (s, 1H), 7.89 (d, J = 2.7 Hz, 1H), 7.80 (dd, J = 8.4, 3.0 Hz, 3H), 7.75-7.64 (m, 3H), 7.21 (d, J = 8.9 Hz, 1H), 7.16 (d, J = 2.0 Hz, 1H), 7.02 (dd, J = 8.6, 1.9 Hz, 1H), 3.79 (s, 3H), 3.22-3.13 (m, 2H), 2.98 (t, J = 5.3 Hz, 4H), 1.54 (t, J = 5.8 Hz, 4H), 1.21 (t, J = 7.3 Hz, 3H), 0.35 (s, 4H). |
| 15 | | |
| 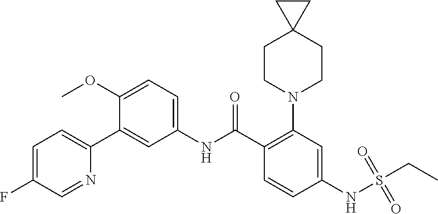 [SMILES: CCS(=O)(=O)Nc1ccc(C(=O)Nc2ccc(OC)c(-c3ccc(F)cn3)c2)c(N2CCC3(CC2)CC3)c1] | 539.3 [M + 1] | NA |
| 16 | | |

### BIOLOGICAL EVALUATION

### Test Example 1. Determination of the Inhibitory Effects of the Compound of the Present Invention on Proliferation of OVCAR-3 Cells

The following method was used to determine the effect of the compound of the present invention on the proliferation of OVCAR-3 cells. OVCAR-3 cells (comprising TP53 R248Q mutation) were purchased from the Cell Resource Center of Shanghai Institute of Life Sciences, Chinese Academy of Sciences, and cultured in a RPMI 1640 medium comprising10% fetal bovine serum, 100 U penicillin and 100 μg/mL streptomycin. Cell viability was determined by Cell Titer-Glo® Luminescent Cell Viability Assay Kit (Promega, article No. G7573).

An experimental method was carried out according to steps provided in the kit manual, with details as follows: firstly, a test compound was dissolved in DMSO to prepare a 10 mM storage solution, and then diluted with a culture medium to prepare a test sample. A final concentration of the compound ranged from 1000 nM to 0.015 nM. Cells in a logarithmic growth phase were inoculated into a 96-mesh cell culture plate at a density of 1000 cells per mesh, cultured overnight in a 5% CO₂ incubator at 37° C., and then cultured for 72 hours after adding the test compound. After completion of cultivation, 50 uL CellTiter-Glo detection solution was added into each mesh, shook for 5 minutes, and stood for 10 minutes, and then a luminous value of each mesh of the sample was read by a microplate reader using Luminescence mode. Compared with the numerical value of a control group (0.3% DMSO), a percentage inhibition rate of the compound at each concentration point was calculated, and then an IC₅₀ value of the compound inhibiting cell proliferation was obtained by a nonlinear regression analysis with a logarithmic-inhibition rate of the compound concentration in a GraphPad Prism 5 software, as shown in Table 1.

TABLE 1
IC₅₀ data of the compound of the present invention
inhibiting the proliferation of OVCAR-3 cells

| Example No. | IC₅₀(nM) |
|---|---|
| Control compound AMG 650 | 64.5 |
| 6 | 1.5 |
| 7 | 1.9 |
| 8 | 17.4 |
| 9 | 1.5 |
| 10 | 7.5 |
| 11 | 14.7 |
| 16 | 4.5 |

Conclusion: the compound of the present invention had a good inhibitory effect on the proliferation of OVCAR-3 cells, preferably IC₅₀<50 nM.

Remarks: structure of AMG 650 (prepared according to Example 4 of the published patent WO2020132648A1) was as follows:

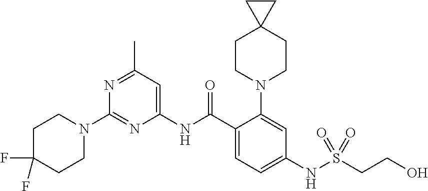 [SMILES: Cc1cc(NC(=O)c2ccc(NS(=O)(=O)CCO)cc2N2CCC3(CC2)CC3)nc(N2CCC(F)(F)CC2)n1]

### Test Example 2. Determination of the Inhibitory Effects of the Compound of the Present Invention on Proliferation of HT-29 Cells

The following method was used to determine the effect of the compound of the present invention on the proliferation of HT-29 cells. HT-29 cells (comprising TP53 R273H mutation) were purchased from the Cell Resource Center of Shanghai Institute of Life Sciences, Chinese Academy of Sciences, and cultured in a McCOY's 5A medium comprising 10% fetal bovine serum, 100 U penicillin and 100 μg/mL streptomycin. Cell viability was determined by Cell Titer-Glo® Luminescent Cell Viability Assay Kit (Promega, article No. G7573).

An experimental method was carried out according to steps provided in the kit manual, with details as follows: firstly, a test compound was dissolved in DMSO to prepare a 10 mM storage solution, and then diluted with a culture medium to prepare a test sample. A final concentration of the compound ranged from 1000 nM to 0.015 nM. Cells in a logarithmic growth phase were inoculated into a 96-mesh cell culture plate at a density of 1000 cells per mesh, and cultured overnight in a 5% CO₂ incubator at 37° C., and then cultured for 120 hours after adding the test compound. After completion of cultivation, 50 uL CellTiter-Glo detection solution was added into each mesh, shook for 5 minutes, and stood for 10 minutes, and then a luminous value of each mesh of the sample was read by a microplate reader using Luminescence mode. Compared with a numerical value of a control group (0.3% DMSO), a percentage inhibition rate of the compound at each concentration point was calculated, and then an IC₅₀ value of the compound inhibiting cell proliferation was obtained by a nonlinear regression analysis with a logarithmic-inhibition rate of the compound concentration in a GraphPad Prism 5 software, as shown in Table 2.

TABLE 2
IC₅₀ data of the compound of the present invention
inhibiting the proliferation of HT-29 cells

| Example No. | IC₅₀(nM) |
|---|---|
| Control compound AMG 650 | 61.7 |
| 6 | 3.2 |
| 7 | 52.4 |
| 8 | 14.5 |
| 9 | 3.3 |
| 10 | 42.8 |
| 11 | 3.9 |
| 16 | 27.1 |

Conclusion: the compound of the present invention had a relatively good inhibitory effect on the proliferation of HT-29 cells, preferably IC₅₀<50 nM.

### Test Example 3. Test of the Inhibitory Effects of the Compound of the Present Invention on KIF18A Enzyme Activity

The following method was used to determine a degree of inhibition of the compound of the present invention on the activity of recombinant human KIF18A enzyme in vitro. The method used ADP-Glo™ Kinase Assay kit (article No. V9102) of Promega Company. For detailed experimental operation, please refer to the kit instructions.

The experimental procedure was briefly described as follows: firstly, the test compound was dissolved in DMSO to prepare a storage solution, and then diluted by gradient using a reaction buffer A (15 mM Tris, pH 7.5, 10 mM MgCl₂, 0.01% Pluronic F-68), and a final concentration of the test compound in a reaction system ranged from 10000 nM to 0.15 nM; a KIF18A protein and ATP working solution were prepared with a reaction buffer B (15 mM Tris, pH 7.5, 10 mM MgCl₂, 0.01% Pluronic F-68, 37.5 ug/ml tubulin, 1.25 uM paclitaxel). The reaction was carried out in a 384-mesh microplate. Firstly, the compound and recombinant human KIF18A protein (final concentration of 100 nM, entrusted to Genscript for expression) were added into the mesh, and incubated at room temperature for 20 minutes. Then, ATP solution (component V915A from ADP-Glo™ Kinase Assay kit, final concentration of 60 uM) was added to the reaction solution, and the mixture was incubated at room temperature for 20 minutes. Subsequently, 5 μL ADP-Glo Reagent was added to the reaction system and the obtained system was incubated at troom temperature for 50 minutes. After that, 10 μL of Kinase Detection Reagent was added to the reaction system and the obtained system was incubated at room temperature for 30 minutes. After the completion of incubation, a chemiluminescence intensity value of each mesh was measured by a microplate reader using Luminescence mode. Compared with a numerical value of a control group (0.1% DMSO), a percentage inhibition rate of the compound at each concentration point was calculated, and then an IC₅₀ value of the compound inhibiting cell proliferation was obtained by a nonlinear regression analysis with a logarithmic-inhibition rate of the compound concentration in a GraphPad Prism 5 software, as shown in Table 3.

TABLE 3
IC₅₀ data of the compound of the present invention
inhibiting KIF18A enzyme activity

| Example No. | IC₅₀(nM) |
|---|---|
| Control compound AMG 650 | 173 |
| 7 | 177 |
| 8 | 97.8 |
| 11 | 145 |
| 13 | 137 |
| 16 | 185 |

Conclusion: the compound of the present invention had a good inhibitory effect on the inhibition of KIF18A enzyme activity, preferably IC₅₀<200 nM.

### Test Example 4. Pharmacokinetic Test of the Compound of the Present Invention in Mice

### 1. Experimental Purpose

ICR mice were used as test animals, and drug concentrations in plasma at different time points were determined by a LC/MS/MS method after gavage administration of the control compound AMG 650 and compound 9 of the present invention to study the pharmacokinetic characteristics of the compound of the present invention in mice.

### 2. Experimental Scheme

### 2.1 Experimental Drugs and Animals;

Control Compound AMG650, Compound 9;

ICR mice, male, 27.3-30.3 g, purchased from Charles River Experimental Animal Technology Co., Ltd.

### 2.2 Drug Preparation

A proper amount of pharmaceutical compound was weighed, a proper amount of DMA, CrEL and 5% GS were added in turn, then the system was mixed evenly by ultrasonic vortex to prepare a 1 mg/mL drug administration preparation, wherein DMA, CrEL and 5% GS=10:10:80 (v:v:v).

### 2.3 Drug Administration

ICR mice, each compound injection group (9 mice in each group) to be determined, were fasted overnight, fed was given 4 hours after gavage administration (PO, the dosage of the compound was 10 mg/kg, the dosage volume was 10 mL/kg).

### 3. Operation

About 0.1 mL blood was collected from orbits 0.25 hours, 0.5 hours, 1 hour, 2 hours, 4 hours, 8 hours, 10 hours and 24 hours before and after drug administration, and a whole blood sample was placed in an anticoagulant tube including EDTA-K2. Blood samples were collected and then placed on ice, and plasma was separated by centrifugation (centrifugation condition: 1500 g, 10 minutes). The collected plasma was stored at −40° C. to −20° C. before analysis.

LC-MS/MS was used to determine the content of compounds to be tested in plasma of mice after gavage administration.

### 4. Results of Pharmacokinetic Parameters

The pharmacokinetic parameters of the compound of the present invention can be seen from Table 4 below.

TABLE 4
Pharmacokinetic parameters of the compound of the present invention in mice

| | Pharmacokinetic experiment | | | |
|---|---|---|---|---|
| | Mode of | Blood | Curve area | Half-life |
| | administration | concentration | AUC_{0-∞} | period |
| Compound No. | (dosage) | Cmax (ng/mL) | (ng · h/mL) | T½ (h) |
| Control compound AMG650 | Oral (10 mg/mL) | 3050 | 52500 | 9.64 |
| Example 9 | Oral (10 mg/mL) | 5269 | 52159 | 5.9 |

Conclusion: the blood drug concentration and the area under a curve of compound 9 of the present invention were both relatively high, so that the compound had a favorable pharmacokinetic property.

1. A compound or a stereoisomer or a tautomer thereof or a pharmaceutically acceptable salt thereof as shown by general formula (I): 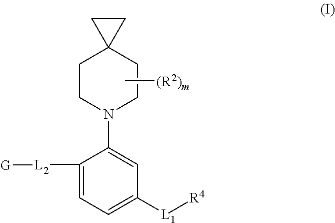
wherein,
G is selected from 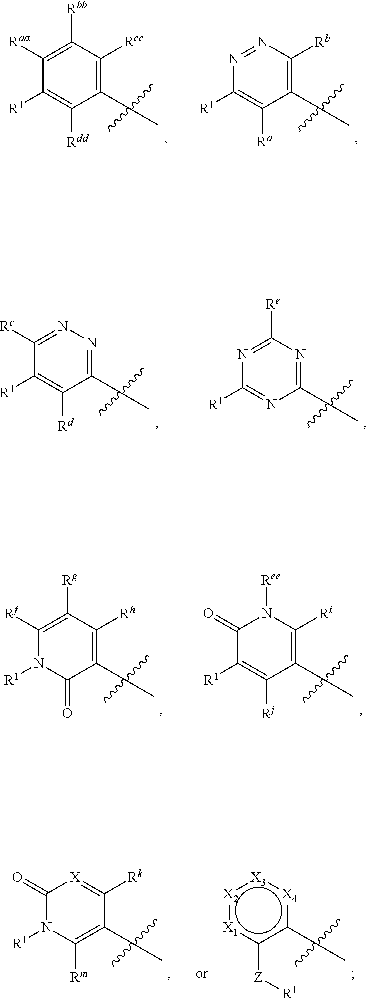
R^{aa}, R^{bb}, R^{cc} and R^{dd} are the same or different and are each independently selected from hydrogen atom, hydroxyl, cyano, amino, alkoxy or haloalkoxy, and R^{aa}, R^{bb}, R^{cc} and R^{dd} are not hydrogen atoms simultaneously;
R^{a}, R^{b}, R^{c}, R^{d}, R^{e}, R^{f}, R^{g}, R^{h}, Rⁱ, R^{j}, R^{k} and R^{m} are the same or different and are each independently selected from hydrogen atom, halogen, hydroxyl, cyano, alkyl or alkoxy; wherein the alkyl or alkoxy group is optionally further substituted by one or more substituents selected from halogen, hydroxyl, cyano, alkyl or alkoxy;
R^{cc} is selected from hydrogen atom or alkyl;
X is selected from N or CR^{ff}, and R^{ff} is selected from hydrogen atom, halogen, hydroxyl, cyano, alkyl, alkoxy, haloalkyl or haloalkoxy;
X₁, X₂, X₃ and X₄ are each independently selected from CR^{gg} or N atom, and at most two atoms in X₁, X₂, X₃ and X₄ are N atoms at the same time, and R^{gg} is selected from hydrogen atom, halogen, hydroxyl, cyano, alkyl, alkoxy, haloalkyl or haloalkoxy;
Z is selected from a bond, —C(O)— or S(O)ᵣ;
L₁ is selected from a bond or C₁-C₆ alkylene, wherein the alkylene is optionally further substituted by one or more substituents selected from halogen, hydroxyl, cyano or alkoxy, and wherein one or more methylenes of the alkylenes are optionally substituted by one or more O, S(O)ᵣ, C(O) or NR^{A};
R^{A} is selected from hydrogen atom or alkyl;
L₂ is selected from 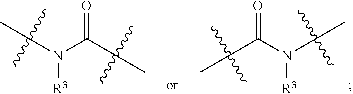
R³ is each independently selected from hydrogen atom or alkyl, wherein the alkyl is optionally further substituted by one or more substituents selected from halogen, hydroxyl, cyano or alkoxy; R³ is preferably a hydrogen atom;
R¹ is each independently selected from hydrogen atom, cyano, halogen, alkyl, cycloalkyl, heterocyclyl, aryl, heteroaryl, —OR⁵, —C(O)R⁵, —C(O)OR⁵, —NHC(O)R⁵, —NHC(O)OR⁵, —NR⁶R⁷, —C(O)NR⁶R⁷, —CH₂NHC(O)OR⁵, —CH₂NR⁶R⁷ or —S(O)ᵣR⁵, wherein the alkyl, cycloalkyl, heterocyclyl, aryl or heteroaryl is optionally further substituted by one or more substituents selected from hydroxyl, halogen, nitro, cyano, alkyl, alkoxy, haloalkyl, haloalkoxy, cycloalkyl, halocycloalkyl, heterocyclyl, haloheterocyclyl, aryl, heteroaryl, haloheteroaryl, ═O, —C(O)R⁸, —C(O)OR⁸, —OC(O)R⁸, —NR⁹R¹⁰, —C(O)NR⁹R¹⁰, —SO₂NR⁹R¹⁰ or —NR⁹C(O)R¹⁰;
R² is the same or different and is each independently selected from halogen, hydroxyl, cyano, alkyl or alkoxy; wherein alkyl or alkoxy is optionally further substituted by one or more substituents selected from halogen, hydroxyl, cyano, alkyl or alkoxy;
R⁴ is selected from hydrogen atom, cyano, halogen, alkyl, hydroxyl, cycloalkyl, heterocyclyl, aryl or heteroaryl; wherein alkyl, cycloalkyl, heterocyclyl, aryl or heteroaryl is optionally further substituted by one or more substituents selected from hydroxyl, halogen, nitro, cyano, alkyl, alkoxy, haloalkyl, haloalkoxy, cycloalkyl, heterocyclyl, aryl, heteroaryl, ═O, —C(O)R⁸, —C(O)OR⁸, —OC(O)R⁸, —NR⁹R¹⁰, —C(O)NR⁹R¹⁰, —SO₂NR⁹R¹⁰ or —NR⁹C(O)R¹⁰;
R⁵ is each independently selected from hydrogen atom, alkyl, cycloalkyl, heterocyclyl, aryl or heteroaryl; wherein alkyl, cycloalkyl, heterocyclyl, aryl or heteroaryl is optionally further substituted by one or more substituents selected from hydroxyl, halogen, nitro, cyano, alkyl, alkoxy, haloalkyl, haloalkoxy, cycloalkyl, heterocyclyl, aryl, heteroaryl, ═O, —C(O)R⁸, —C(O)OR^{B}, —OC(O)R⁸, —NR⁹R¹⁰, —C(O)NR⁹R¹⁰, —SO₂NR⁹R¹⁰ or —NR⁹C(O)R¹⁰;
R⁶ and R⁷ are each independently selected from hydrogen atom, hydroxyl, halogen, alkyl, alkoxy, cycloalkyl, heterocyclyl, aryl or heteroaryl, wherein alkyl, alkoxy, cycloalkyl, heterocyclyl, aryl or heteroaryl is optionally further substituted by one or more substituents selected from hydroxyl, halogen, nitro, cyano, alkyl, alkoxy, cycloalkyl, heterocyclyl, aryl, heteroaryl, ═O, —C(O)R⁸, —C(O)OR⁸, —OC(O)R⁸, —NR⁹R¹⁰, —C(O)NR⁹R¹⁰, —SO₂NR⁹R¹⁰ or —NR⁹C(O)R¹⁰;
or, R⁶ and R⁷ and the atoms attached thereto form a 4-8-membered heterocyclyl group, wherein the 4-8-membered heterocyclyl group comprises one or more N, O or S(O)ᵣ, and the 4-8-membered heterocyclyl group is optionally further substituted by one or more substituents selected from hydroxyl, halogen, nitro, cyano, alkyl, alkoxy, cycloalkyl, heterocyclyl, aryl, heteroaryl, ═O, —C(O)R⁸, —C(O)OR⁸, —OC(O)R⁸, —NR⁹R¹⁰, —C(O)NR⁹R¹⁰, —SO₂NR⁹R¹⁰ or —NR⁹C(O)R¹⁰;
R⁸, R⁹ and R¹⁰ are each independently selected from hydrogen atom, alkyl, amino, cycloalkyl, heterocyclyl, aryl or heteroaryl, wherein alkyl, cycloalkyl, heterocyclyl, aryl or heteroaryl is optionally further substituted by one or more substituents selected from hydroxyl, halogen, nitro, amino, cyano, alkyl, alkoxy, cycloalkyl, heterocyclyl, aryl, heteroaryl, carboxyl or carboxylate;
m is 0, 1 or 2; m is preferably 0; and
r is each independently 0, 1 or 2.

2. The compound, or the stereoisomer or the tautomer thereof or the pharmaceutically acceptable salt thereof according to claim 1, wherein the compound or the stereoisomer or the tautomer thereof or the pharmaceutically acceptable salt is a compound, or a stereoisomer, a tautomer or a pharmaceutically acceptable salt as shown by general formula (II) or general formula (III): 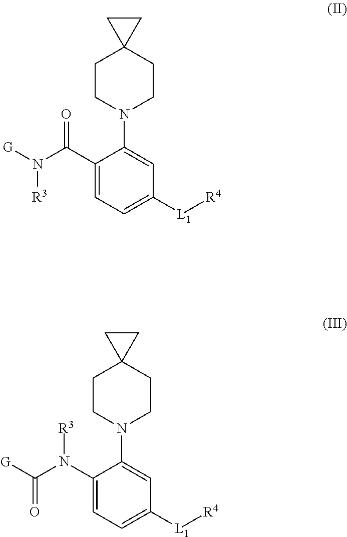
wherein, G, R³, R⁴ and L₁ are as defined in claim 1.

3. The compound, or the stereoisomer or the tautomer thereof or the pharmaceutically acceptable salt thereof according to claim 1, wherein G is selected from 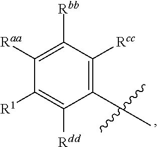
wherein,
R^{aa}, R^{bb}, R^{cc} and R^{dd} are selected from hydrogen atom, hydroxyl, cyano, amino group, methoxy or trifluoromethoxy; and
R¹ is selected from phenyl, 5-6-membered heteroaryl, 3-10-membered heterocyclyl or C₃-C₁₀ cycloalkyl, wherein the phenyl, 5-6-membered heteroaryl, 3-10-membered heterocyclyl or C₃-C₁₀ cycloalkyl is optionally further substituted by one or more substituents selected from halogen, alkyl, haloalkyl, alkoxy or haloalkoxy;
preferably, wherein R¹ is selected from

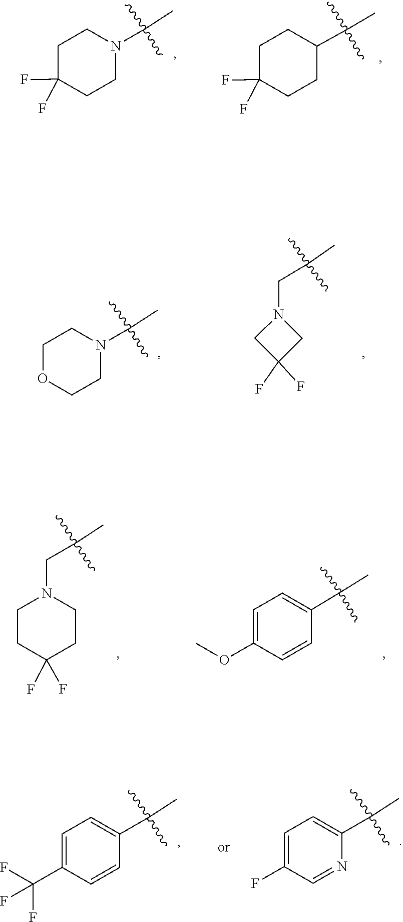

4. The compound, or the stereoisomer or the tautomer thereof or the pharmaceutically acceptable salt thereof according to claim 1, wherein G is selected from 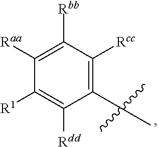
wherein,
R^{aa}, R^{bb}, R^{cc} and R^{dd} are selected from hydrogen atom, hydroxyl, cyano, amino, methoxy or trifluoromethoxy; and
R¹ is selected from C₁₋₃ alkyl, wherein the C₁₋₃ alkyl is optionally further substituted by one or more substituents selected from cycloalkyl, halocycloalkyl, heterocyclyl, haloheterocyclyl, aryl, heteroaryl or haloheteroaryl
preferably, wherein R¹ is selected from

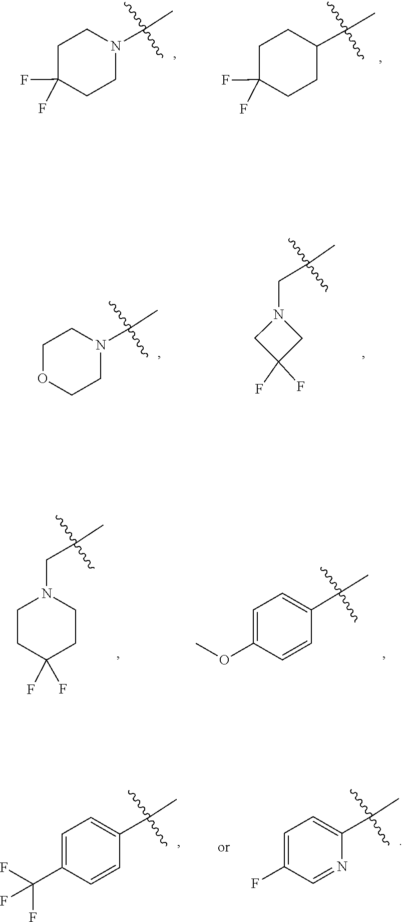

5. (canceled)

6. The compound, or the stereoisomer or the tautomer thereof or the pharmaceutically acceptable salt thereof according to claim 1, wherein G is selected from 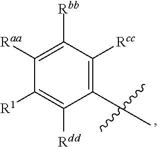
wherein,
R^{aa}, R^{bb}, R^{cc} and R^{dd} are selected from hydrogen atom, hydroxyl, cyano, amino or methoxy; and
R¹ is selected from 3-10-membered heterocyclyl or C₃-C₁₀ cycloalkyl, wherein the heterocyclyl or cycloalkyl is optionally further substituted by one or more halogens;
preferably, wherein R¹ is

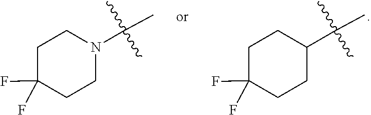 [SMILES: *N1CCC(F)(F)CC1 ; *C1CCC(F)(F)CC1]

7. The compound, or the stereoisomer or the tautomer thereof or the pharmaceutically acceptable salt thereof according to claim 1, wherein G is selected from 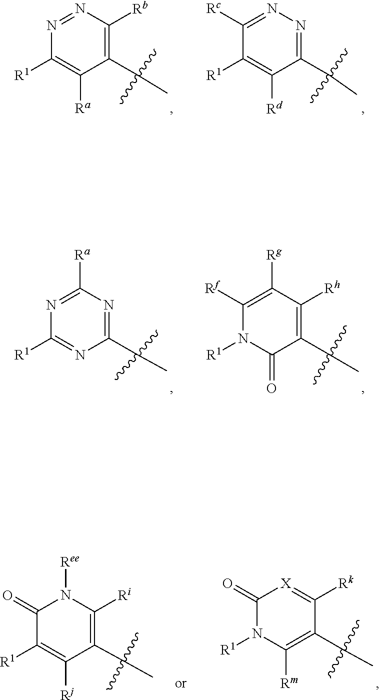
wherein,
R^{a}, R^{b}, R^{c}, R^{d}, R^{e}, R^{f}, R^{g}, R^{h}, Rⁱ, R^{j}, R^{k} and R^{m} are the hydrogen atoms;
X is selected from N or CR^{ff}, R^{ee} and R^{ff} are independently selected from hydrogen atom or methyl; and
R¹ is each independently selected from 3-10-membered heterocyclyl or C₃-C₁₀ cycloalkyl, wherein the heterocyclyl or cycloalkyl is optionally further substituted by one or more halogens;
preferably, wherein R¹ is

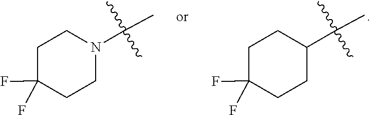 [SMILES: *N1CCC(F)(F)CC1 ; *C1CCC(F)(F)CC1]

8. The compound, or the stereoisomer or the tautomer thereof or the pharmaceutically acceptable salt thereof according to claim 1, wherein G is selected from 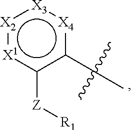
wherein,
Z is —C(O)—;
X₁, X₂, X₃ and X₄ are each independently selected from CR^{gg} or N atom, and at most two atoms in X₁, X₂, X₃ and X₄ are N atoms at the same time, and R^{gg} is a hydrogen atom; and
R¹ is selected from 3-10-membered heterocyclyl or C₃-C₁₀ cycloalkyl, wherein the heterocyclyl or cycloalkyl is optionally further substituted by one or more halogens;
preferably, wherein R¹ is

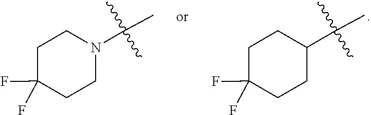 [SMILES: *N1CCC(F)(F)CC1 ; *C1CCC(F)(F)CC1]

9. (canceled)

10. The compound, or the stereoisomer or the tautomer thereof or the pharmaceutically acceptable salt thereof according to claim 1, wherein
L₁ is selected from a bond or C₁-C₆ alkylene, wherein the alkylene is optionally further substituted by one or more hydroxyl, and wherein one or more methylene of the alkylene are optionally substituted by one or more O, S(O)ᵣ, C(O) or NR^{b};
r is 2; and
R^{b} is selected from hydrogen atom or methyl.

11. The compound, or the stereoisomer or the tautomer thereof or the pharmaceutically acceptable salt thereof according to claim 10, wherein L₁ is selected from a bond, —NHSO₂CH₂CH₂—, —SO₂NHCH₂CH₂—, —SO₂—, —CH₂SO₂—, —NHSO₂—, —SO₂NH—, —NHC(CH₃)₂CH₂—, —C(O)NHCH₂CH₂—, —C(O)NHC(CH₃)₂CH₂—, —C(O)N(CH₃)CH₂CH₂—, —CH(CH₃)(OH)CH₂—, —NHSO₂CH(CH₃)CH₂—, —SO₂NHC(CH₃)₂CH₂—, —C(O)NH—, —NHCH₂CH₂ or —CH₂SO₂CH₂CH₂—.

12. The compound, or the stereoisomer or the tautomer thereof or the pharmaceutically acceptable salt thereof according to claim 1, wherein R⁴ is selected from hydrogen atom, hydroxyl, alkyl, heterocyclyl, cycloalkyl or heteroaryl, wherein the alkyl, heterocyclyl, cycloalkyl or heteroaryl is optionally further substituted by one or more substituents selected from hydroxyl or alkyl.

13. The compound, or the stereoisomer or the tautomer thereof or the pharmaceutically acceptable salt thereof according to claim 1, wherein

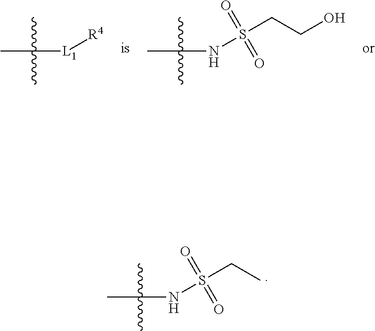

14. The compound, or the stereoisomer or the tautomer thereof or the pharmaceutically acceptable salt thereof according to claim 1, wherein R³ is a hydrogen atom.

15. The compound, or the stereoisomer or the tautomer thereof or the pharmaceutically acceptable salt thereof according to claim 1, wherein the compound is

[chemical structure]

15-20. (canceled)

21. A pharmaceutical composition, wherein the pharmaceutical composition comprises an effective dose of the compound, the stereoisomer or the tautomer thereof or the pharmaceutically acceptable salt thereof according to claim 1, and a pharmaceutically acceptable carrier, an excipient or a composition thereof.

22. A method for inhibiting KIF18A, comprising administering a therapeutically effective amount of the compound, the stereoisomer or the tautomer thereof or the pharmaceutically acceptable salt thereof according to claim 1, or a pharmaceutical composition the compound, the stereoisomer or the tautomer thereof or the pharmaceutically acceptable salt thereof according to claim 1 to a subject in need thereof.

23. A method for treating a disease mediated by KIF18A, comprising administering a therapeutically effective amount of the compound, the stereoisomer or the tautomer thereof or the pharmaceutically acceptable salt thereof according to claim 1, or a pharmaceutical composition the compound, the stereoisomer or the tautomer thereof or the pharmaceutically acceptable salt thereof according to claim 1 to a subject in need thereof.

24. The method according to claim 23, wherein the disease mediated by KIF18A is cancer.

25. The method according to claim 24, wherein the cancer is selected from hepatocellular carcinoma, glioblastoma, colon cancer, breast cancer, lung cancer, cholangiocarcinoma, pancreatic cancer, prostate cancer, bladder cancer, head cancer, neck cancer, cervical cancer, ovarian cancer, synovial sarcoma, rhabdomyosarcoma, colorectal cancer and lung adenocarcinoma.

26. A method for treating cancer, comprising administering a therapeutically effective amount of the compound, the stereoisomer or the tautomer thereof or the pharmaceutically acceptable salt thereof according to claim 1, or a pharmaceutical composition the compound, the stereoisomer or the tautomer thereof or the pharmaceutically acceptable salt thereof according to claim 1 to a subject in need thereof.

27. The method according to claim 24, wherein the cancer is selected from hepatocellular carcinoma, glioblastoma, colon cancer, breast cancer, lung cancer, cholangiocarcinoma, pancreatic cancer, prostate cancer, bladder cancer, head cancer, neck cancer, cervical cancer, ovarian cancer, synovial sarcoma, rhabdomyosarcoma, colorectal cancer and lung adenocarcinoma.
